(19) United States
(12) Patent Application Publication
(10) Pub. No.: US 2022/0168307 A1
(43) Pub. Date: Jun. 2, 2022

(54) TREATMENT OF SMA

(71) Applicant: Hoffmann-La Roche Inc., Little Falls, NJ (US)

(72) Inventor: Heidemarie Kletzl, Basel (CH)

(73) Assignee: Hoffmann-La Roche Inc., Little Falls, NJ (US)

(21) Appl. No.: 17/548,352

(22) Filed: Dec. 10, 2021

Related U.S. Application Data
(63) Continuation of application No. PCT/EP2020/066003, filed on Jun. 10, 2020.

(30) Foreign Application Priority Data
Jun. 12, 2019 (EP) 19179563.2

(51) Int. Cl. A61K 31/519; A61K 31/5517

(52) U.S. Cl. CPC A61K 31/519; A61K 9/0053; A61K 31/5517

(57) ABSTRACT

The present invention relates to 7-(4,7-diazaspiro[2.5]octan-7-yl)-2-(2,8-dimethylimidazo[1,2-b]pyridazin-6-yl)pyrido[1,2-a]pyrimidin-4-one for use in the treatment of spinal muscular atrophy (SMA), its pharmaceutical composition to be used in the treatment of SMA, its methods of treatment thereof.

### CROSS-REFERENCE TO RELATED APPLICATIONS

This application is a continuation of International Application No. PCT/EP2020/066003 having an International filing date of Jun. 10, 2020, which claims benefit of and priority to European Patent Application No. EP19179563.2, filed Jun. 12, 2019, the contents of each which are incorporated herein by reference in their entirety.

### FIELD OF INVENTION

The invention relates to 7-(4,7-diazaspiro[2.5]octan-7-yl)-2-(2,8-dimethylimidazo[1,2-b]pyridazin-6-yl)pyrido[1,2-a]pyrimidin-4-one for use in the treatment of spinal muscular atrophy (SMA), its pharmaceutical composition to be used in the treatment of SMA, its methods of treatment thereof.

More particularly, the invention herein disclosed is based on the surprising finding of a drug-drug interaction between 7-(4,7-diazaspiro[2.5]octan-7-yl)-2-(2,8-dimethylimidazo[1,2-b]pyridazin-6-yl)pyrido[1,2-a]pyrimidin-4-one, also known as risdiplam, and molecules which are CYP3A substrates, more particularly wherein the CYP3A substrate is midazolam.

Spinal muscular atrophy (SMA), in its broadest sense, describes a collection of inherited and acquired central nervous system (CNS) diseases characterized by progressive motor neuron loss in the spinal cord and brainstem causing muscle weakness and muscle atrophy. SMA is characterized by a degeneration of the alpha motor neurons from the anterior horn of the spinal cord leading to muscular atrophy and resulting in paralysis. This alpha motor neuron degeneration thus substantially compromises the vital prognosis of patients. In healthy subjects, these neurons transmit messages from the brain to the muscles, leading to the contraction of the latter. In the absence of such a stimulation, the muscles atrophy. Subsequently, in addition to a generalized weakness and atrophy of the muscles, and more particularly of those of the trunk, upper arms and thighs, these disorders can be accompanied by serious respiratory problems.

Infantile SMA is the most severe form of this neurodegenerative disorder. Symptoms include muscle weakness, poor muscle tone, weak cry, limpness or a tendency to flop, difficulty sucking or swallowing, accumulation of secretions in the lungs or throat, feeding difficulties, and increased susceptibility to respiratory tract infections. The legs tend to be weaker than the arms and developmental milestones, such as lifting the head or sitting up, cannot be reached. In general, the earlier the symptoms appear, the shorter the lifespan. As the motor neuron cells deteriorate, symptoms appear shortly afterward. The severe forms of the disease are fatal and all forms have no known cure. The course of SMA is directly related to the rate of motor neuron cell deterioration and the resulting severity of weakness. Infants with a severe form of SMA frequently succumb to respiratory complications due to weakness in the muscles that support breathing. Children with milder forms of SMA live much longer, although they may need extensive medical support, especially those at the more severe end of the spectrum. The clinical spectrum of SMA disorders has been divided into the following five groups:
1) Type 0 SMA (In Utero SMA) is the most severe form of the disease and begins before birth. Usually, the first symptom of Type 0 SMA is reduced movement of the fetus that can first be observed between 30 and 36 weeks of pregnancy. After birth, these newborns have little movement and have difficulties with swallowing and breathing and die shortly after birth.
2) Type I SMA (Infantile SMA or Werdnig-Hoffmann disease) presents symptoms between 0 and 6 months; this form of SMA is very severe. Patients never achieve the ability to sit, and death usually occurs within the first 2 years.
3) Type II SMA (Intermediate SMA) has an age of onset at 7-18 months. Patients achieve the ability to sit unsupported, but never stand or walk unaided. Prognosis in this group is largely dependent on the degree of respiratory involvement.
4) Type III SMA (Juvenile SMA or Kugelberg-Welander disease) is generally diagnosed after 18 months. Type 3 SMA individuals are able to walk independently at some point during their disease course but often become wheelchair-bound during youth or adulthood.
5) Type IV SMA (Adult onset SMA). Weakness usually begins in late adolescence in the tongue, hands, or feet, then progresses to other areas of the body. The course of adult SMA is much slower and has little or no impact on life expectancy.

All the forms of spinal muscular atrophy are accompanied by progressive muscle weakness and atrophy subsequent to the degeneration of the neurons from the anterior horn of the spinal cord. SMA currently constitutes one of the most common causes of infant mortality. It equally affects girls or boys in all regions of the world with a prevalence of between 1/6000 and 1/10 000.

There is currently no approved oral treatment for SMA that provides stabilization or improvement of motor function. Several drug candidates are currently under investigation in the nonclinical and clinical settings (Lewelt A, et al, Curr Neurol Neurosci Rep. 2012; 12:42-532; Arnold et al., Muscle Nerve. 2015; 51:157-67). Nusinersen, an intrathecally-administered antisense oligonucleotide which promotes the inclusion of exon 7 in SMN2 pre mRNA, has received approvals in the US, EU and other jurisdictions. Recently, onasemnogene abeparvovac-xio has received approval in the US as an intravenously administered gene therapy.

Despite a better understanding of the genetic basis and pathophysiology of SMA, and the several strategies for treatment having been explored, none have yet demonstrated success as an oral treatment in the clinic. The present invention intends to respond to this oral treatment need. 7-(4,7-diazaspiro[2.5]octan-7-yl)-2-(2,8-dimethylimidazo[1,2-b]pyridazin-6-yl)pyrido[1,2-a]pyrimidin-4-one is currently investigated in clinical phase II/III.

A phase I, 2-part, open-label study to investigate the safety and tolerability of multiple doses of 7-(4,7-diazaspiro[2.5]octan-7-yl)-2-(2,8-dimethylimidazo[1,2-b]pyridazin-6-yl)pyrido[1,2-a]pyrimidin-4-one, also known under its INN name risdiplam, and the effect of risdiplam on the pharmacokinetics of midazolam following oral administration (BP41361) is being performed in healthy participants aged 18 to 55 years.

Risdiplam did not show any significant reversible or time-dependent inhibition of CYPs 1A2, 2B6, 2C8, 2C9, 2C19, or 2D6 in-vitro, but it was surprisingly found that risdiplam shows time-dependent inhibition of CYP3A4/5.

The time-dependent inhibition of CYP3A4/5 by risdiplam may expose patients to an overdose of drugs being metabolized by CYP3A, such as midazolam. Patients being exposed at higher than the usual therapeutic exposure level of these drugs metabolized by CYP3A may encounter undesirable adverse events, which in some instances may result in severe adverse events. In particular, the adverse events of being over-exposed to midazolam may result in sedation, somnolence, confusion, impaired coordination, diminished reflexes, effects on vital signs, respiratory depression and respiratory arrest, coma, and in the worst case death.

Midazolam is rapidly absorbed after oral administration and is subject to substantial intestinal and hepatic first-pass metabolism. Midazolam is primarily metabolized in the liver and gut by human CYP3A to its pharmacologically active metabolite 1-OH-midazolam. In the subsequent UDP-glucuronosyltransferase-mediated phase II-reaction, the main urinary metabolite 1′-OH-midazolam-glucuronide is formed; 63% to 80% of the dose is found conjugated in the urine within 24 hours, while only 1% is excreted unchanged. The mean t¹² of midazolam ranges from 2.2 to 6.8 hours following single oral dose administration.

The PK interactions with CYP3A inhibitors or inducers are of higher magnitude on oral administration of midazolam compared to intravenous administration, particularly because CYP3A is also present in the upper GI tract and by the oral administration route, both systemic clearance and bioavailability are subject to change, while by the parenteral administration route, only the systemic clearance will be affected.

Pharmacodynamic properties of midazolam and its metabolites include sedative, anxiolytic, amnesic, and hypnotic activities. Benzodiazepine pharmacological effects appear to result from reversible interactions with the γ-amino butyric acid benzodiazepine receptor in the central nervous system (CNS), the major inhibitory neurotransmitter in the CNS.

### BRIEF DESCRIPTION OF THE FIGURES

FIG. 1: Percent Activity Remaining for CYP3A4 (Midazolam). Panel A: risdiplam (RO7034067), Panel B: Positive Control Inhibitor

FIG. 2: Percent Activity Remaining for CYP3A4 (Testosterone). Panel A: risdiplam (RO7034067), Panel B: Positive Control Inhibitor

FIG. 3: Percent Activity Remaining for CYP3A4 (Midazolam). Panel A: risdiplam (RO7034067) plus and minus NADPH, Panel B: Positive Control Inhibitor with NADPH

FIG. 4: Percent Activity Remaining for CYP3A4 (Testosterone). Panel A: risdiplam (RO7034067) plus and minus NADPH, Panel B: Positive Control Inhibitor with NADPH

FIG. 5: Inactivation of CYP3A4 by risdiplam (RO7034067). Panel A: Natural logarithm of the residual activity versus time, Panel B: kobs versus concentration plot

All publications, patent applications, patents and other references mentioned herein are incorporated by reference in their entirety.

The nomenclature used in the present application is based on IPUAC systematic nomenclature, unless indicated otherwise.

Various features and embodiments of the present invention are disclosed herein, however other features of the invention, modifications and equivalents will be apparent to a person skilled in the relevant art, based on the teachings provided. The invention described is not limited to the examples and embodiments provided, various alternatives equivalents will be appreciated by those skilled in the art. As used herein, the singular forms “a”, “an” and “the” include the plural unless the context clearly dictates otherwise. For example, “a” individual will also include “individuals”.

Unless otherwise stated, the following terms used in the specification and claims have the meanings given below:

The term “FMO3” refers to Flavin-containing monooxygenase 3, also known as dimethylaniline monooxygenase [N-oxide-forming] 3 and trimethylamine monooxygenase, with its enzyme commission number (EC number) EC 1.14.13.148, MGI reference 1100496, Cytogenetic location: 1q24.3 and Genomic coordinates (GRCh38): 1:171,090,872-171,117,818

An “individual” or “subject”, used interchangeably, is a mammal. Mammals include, but are not limited to, domesticated animals (e.g., cows, sheep, cats, dogs, and horses), primates (e.g., humans and non-human primates such as monkeys), rabbits, and rodents (e.g., mice and rats). In certain embodiments, the individual or subject is a human. In a particular embodiment of the invention the subject is a human with spinal muscular atrophy (SMA). In another specific embodiment, the subject is a human with SMA caused by an inactivating mutation or deletion in the SMN1 gene on both chromosomes, resulting in a loss of SMN1 gene function.

As used herein, the term “avoid” and forms thereof are contemplated to have as alternatives the terms abstain, desist, forbear, and refrain, and forms thereof. In some cases, the alternative terms will be equivalent. For example, “avoiding” means “refraining from.” Merriam-Webster Online Dictionary, 11^{th} ed., 24 Nov. 2009. As used herein, the term “discontinue” and forms thereof are contemplated to have as alternatives the terms cease, stop, suspend, and quit.

The term “spinal muscular atrophy” (or SMA) relates to a disease caused by an inactivating mutation or deletion in the SMN1 gene on both chromosomes, resulting in a loss of SMN1 gene function. Symptoms of SMA—depending on the type of SMA—include muscle weakness, poor muscle tone, weak cry, weak cough, limpness or a tendency to flop, difficulty sucking or swallowing, difficulty breathing, accumulation of secretions in the lungs or throat, clenched fists with sweaty hand, flickering/vibrating of the tongue, head often tilted to one side, even when lying down, legs that tend to be weaker than the arms, legs frequently assuming a “frog legs” position, feeding difficulties, increased susceptibility to respiratory tract infections, bowel/bladder weakness, lower-than-normal weight, inability to sit without support, failure to walk, failure to crawl, and hypotonia, areflexia, and multiple congenital contractures (arthrogryposis) associated with loss of anterior horn cells.

The term “treating spinal muscular atrophy (SMA)” or “treatment of spinal muscular atrophy (SMA)” includes one or more of the following effects: (i) reduction or amelioration of the severity of SMA; (ii) delay of the onset of SMA; (iii) inhibition of the progression of SMA; (iv) reduction of hospitalization of a subject; (v) reduction of hospitalization length for a subject; (vi) increase of the survival of a subject; (vii) improvement of the quality of life of a subject; (viii) reduction of the number of symptoms associated with SMA; (ix) reduction of or amelioration of the severity of one or more symptoms associated with SMA; (x) reduction of the duration of a symptom associated with SMA; (xi) prevention of the recurrence of a symptom associated with SMA; (xii) inhibition of the development or onset of a symptom of SMA; and/or (xiii) inhibition of the progression of a symptom associated with SMA. More particular, “treating SMA” denotes one or more of the following beneficial effects: (i) a reduction in the loss of muscle strength; (ii) an increase in muscle strength; (iii) a reduction in muscle atrophy; (iv) a reduction in the loss of motor function; (v) an increase in motor neurons; (vii) a reduction in the loss of motor neurons; (viii) protection of SMN deficient motor neurons from degeneration; (ix) an increase in motor function; (x) an increase in pulmonary function; and/or (xi) a reduction in the loss of pulmonary function.

The term “concomitant use” is understood to be interchangeable with concurrent administration or co-administration. Thus, the terms are understood to encompass administration simultaneously or at different times, and by the same route or by different routes, as long as the two agents are given in a manner that allows both agents to be affecting the body at the same time. For example, concomitant use can refer to a medication concomitantly administered, whether prescribed by the same or a different practitioner, or for the same or a different indication. More particularly risdiplam may be administered orally while midazolam may be administered orally, intravenously, via injection into a muscle, intranasal delivery, rectal or through the cheeks.

In detail, “treating SMA” results in the functional ability or helps retain the functional ability for a human infant or a human toddler to sit up unaided or for a human infant, a human toddler, a human child or a human adult to stand up unaided, to walk unaided, to run unaided, to breathe unaided, to turn during sleep unaided, or to swallow unaided.

The term “mg/kg” refers to the dose in milligram of 7-(4,7-diazaspiro[2.5]octan-7-yl)-2-(2,8-dimethylimidazo[1,2-b]pyridazin-6-yl)pyrido[1,2-a]pyrimidin-4-one being used per kilogram of body weight of the subject to be treated. For example, 0.25 mg/kg means a dose of 0.25 milligram of 7-(4,7-diazaspiro[2.5]octan-7-yl)-2-(2,8-dimethylimidazo[1,2-b]pyridazin-6-yl)pyrido[1,2-a]pyrimidin-4-one per kilogram of body weight of the patient to be treated.

The term “patient” refers to a human (such as a male or female human) who has been diagnosed with SMA, in particular that has been diagnosed with SMA and is in need of a therapy that is being metabolized by CYP3A enzymes, more particularly in need of midazolam.

The term “active pharmaceutical ingredient” (or “API”) denotes the compound or molecule in a pharmaceutical composition that has a particular biological activity.

The terms “pharmaceutically acceptable excipient”, “pharmaceutically acceptable carrier” and “therapeutically inert excipient” can be used interchangeably and denote any pharmaceutically acceptable ingredient in a pharmaceutical composition having no therapeutic activity and being non-toxic to the subject administered, such as disintegrators, binders, fillers, solvents, buffers, tonicity agents, stabilizers, antioxidants, surfactants, carriers, diluents or lubricants used in formulating pharmaceutical products.

The term “pharmaceutical composition” refers to a preparation which is in such form as to permit the biological activity of an active ingredient contained therein to be effective, and which contains no additional components which are unacceptably toxic to a subject to which the composition would be administered. The term “pharmaceutically acceptable” denotes an attribute of a material which is useful in preparing a pharmaceutical composition that is generally safe, non-toxic, and neither biologically nor otherwise undesirable and is acceptable for veterinary as well as human pharmaceutical use.

The term “buffer” or “buffer system” denotes a pharmaceutically acceptable excipient or excipient mixture, which stabilizes the pH of a pharmaceutical preparation. Suitable buffers are well known in the art and can be found in the literature. Particular pharmaceutically acceptable buffers comprise citric buffer, malate buffer, maleate buffer, or tartrate buffer, most particularly tartrate buffer. Particular buffer systems of the invention combinations of organic acid and selected salts thereof, e.g. tribasic sodium citrate and citric acid, malic acid and sodium malate, potassium sodium tartrate and tartaric acid, or disodium tartrate and tartaric acid, particularly potassium sodium tartrate and tartaric acid. Alternatively, the organic acid (particularly tartaric acid) can be employed alone as “acidifier” instead of the combination of acid and the corresponding salt. Independently from the buffer used, the pH can be adjusted with an acid or a base known in the art, e.g. hydrochloric acid, acetic acid, phosphoric acid, sulfuric acid and citric acid, sodium hydroxide and potassium hydroxide. Particular acidifier is tartaric acid.

A “pharmaceutically acceptable carrier” refers to an ingredient in a pharmaceutical composition, other than an active ingredient, which is nontoxic to a subject. A pharmaceutically acceptable carrier includes, but is not limited to, a buffer or acidifier, excipient, stabilizer, or preservative.

The term “antioxidant” denotes pharmaceutically acceptable excipients, which prevent oxidation of the active pharmaceutical ingredient. Antioxidants comprise ascorbic acid, glutathione, cysteine, methionine, citric acid, EDTA.

The term “surfactant” denotes a pharmaceutically acceptable excipient which is used to protect protein compositions against mechanical stresses like agitation and shearing. Examples of pharmaceutically acceptable surfactants include poloxamers, polysorbates, polyoxyethylene alkyl ethers (BRIJ®), alkylphenylpolyoxyethylene ethers (TRITON-X@) or sodium dodecyl sulfate (SDS).

The term “poloxamer” denotes non-ionic triblock copolymers composed of a central hydrophobic chain of poly(propylene oxide) (PPO) flanked by two hydrophilic chains of poly(ethylene oxide) (PEO), each PPO or PEO chain can be of different molecular weights. Poloxamers are also known by the trade name Pluronics. Particular Poloxamer is Poloxamer 188, a poloxamer wherein the PPO chain has a molecular mass of 1800 g/mol and a PEO content of 80% (w/w).

The term “polysorbate” denotes oleate esters of sorbitol and its anhydrides, typically copolymerized with ethylene oxide. Particular polysorbates are Polysorbate 20 (poly(ethylene oxide) (20) sorbitan monolaurate, TWEEN 20®) or Polysorbate 80 (poly(ethylene oxide) (80) sorbitan monolaurate, TWEEN 80®).

The “hydrophilic-lipophilic balance” (HLB) value denotes the degree of hydrophilicity of a non-ionic surfactant. The HLB value is determined by the ratio between the molecular mass of the hydrophilic portion of the surfactant molecule and its overall molecular mass, as described by Griffin W. C., Journal of the Society of Cosmetic Chemists (1949) 1:311.

The term “hydrophilic” denotes the capacity of a molecule or portion of a molecule to interact with polar solvents, in particular with water, or with other polar moieties driven by hydrogen bonding, dipole-ion interactions and/or dipole-dipole interactions.

The terms “lipophilic” and “hydrophobic” can be used interchangeably and denote the tendency of a molecule or portion of a molecule to dissolve in non-polar environment such as fats, oils, and non-polar solvents driven by London dispersion forces.

The term “Cₘₐₓ” (expressed in units of ng/mL) means maximum observed plasma concentration.

The term “Tₘₐₓ” (expressed in units of hours, or as a median number of hours for Tₘₐₓ in the study population) means the observed time to reach Cₘₐₓ following drug administration; if it occurs at more than one time point Tₘₐₓ is defined as the first time point with this value.

The term “AUC_{T0-24h}” (expressed in units of ng h/mL) means the area under the plasma concentration time curve (AUC).

The term “sdOCT” refers to spectral domain.optical coherence tomography.

7-(4,7-diazaspiro[2.5]octan-7-yl)-2-(2,8-dimethylimidazo[1,2-b]pyridazin-6-yl)pyrido[1,2-a]pyrimidin-4-one according to the present invention refers to a compound of formula (I)

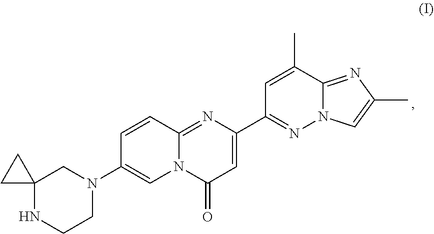 [SMILES: Cc1cn2nc(-c3cc(=O)n4cc(N5CCNC6(CC6)C5)ccc4n3)cc(C)c2n1]

also known as risdiplam, RG7916, RO7034067, CAS Number 1825352-65-5, Methods of making and using the compound are described in EP3143025 A1. Methods of making and using the pharmaceutical composition comprising risdiplam are described in WO2017080967 A1. “CYP3A” refers to the most abundant and most clinically significant subfamily of cytochrome P450 enzymes. The CYP3A subfamily has four human isoforms, 3A4, 3A5, 3A7 and 3A43, CYP3A4 being the most commonly associated with drug interactions. The CYP3A isoforms make up approximately 50% of the liver's total cytochrome P450 and are widely expressed throughout the gastrointestinal tract, kidneys and lungs and therefore are ultimately responsible for the majority of first-pass metabolism. This is important as increases or decreases in first-pass metabolism can have the effect of administering a much smaller or larger dose-equivalent of drug than usual. More than 150 drugs are known substrates of CYP3A4, including many of the opiate analgesics, steroids, antiarrhythmic agents, tricyclic antidepressants, calcium-channel blockers and macrolide antibiotics. CYP3A substrates are for example immunosuppressants (i.e. ciclosporin, tacrolimus, sirolimus), chemotherapeutics (i.e. docetaxel, tamoxifen, paclitaxel, cyclophosphamide, doxorubicin, erlotinib, etoposide, ifosfamide, teniposide, vinblastine, vincristine, vindesine, imatinib, irinotecan, sorafenib, sunitinib, vemurafenib, temsirolimus, anastrozole, gefitinib), azole antifungals (i.e. ketoconazole, itraconazole), macrolides (clarithromycin, erythromycin, telithromycin), antidepressants (i.e amitriptyline, clomipramine, imipramine, cyclobenzaprine, mirtazapine, nefazodone, reboxetine, venlafaxine, trazodone, vilazodone), SSRIs (i.e. citalopram, norfluoxetine, sertraline), antipsychotics (i.e. haloperidol, aripiprazole, risperidone, ziprasidone, pimozide, quetiapine), opioids (i.e. alfentanil, buprenorphine, codeine, fentanyl, hydrocodone, methadone, levacetylmethadol, tramadol), benzodiazepines (i.e.alprazolam, midazolam, triazolam, diazepam, clonazepam), hypnotics (i.e. zopiclone, zaleplon, zolpidem), statins (i.e. atorvastatin, lovastatin, simvastatin, cerivastatin), calcium channel blockers (i.e. diltiazem, felodipine, nifedipine, verapamil), sex hormones agonists and antagonists (i.e. finasteride, estradiol, progesterone, ethinylestradiol, testosterone, toremifene, bicalutamide), H1-receptor antagonists (i.e. terfenadine, astemizole, chlorphenamine), protease inhibitors (i.e. indinavir, ritonavir, saquinavir, nelfinavir) and other drugs (i.e amlodipine, lercanidipine, nitrendipine, nisoldipine, amiodarone, dronedarone, quinidine, sildenafil, tadalafil, kinins, nevirapine, budesonide, hydrocortisone, dexamethasone, albendazole, cisapride, aprepitant, caffeine, cilostazol, dextromethorphan, domperidone, eplerenone, lidocaine, ondansetron, propranolol, salmeterol, warfarin, clopidogrel, omeprazole, nateglinide, methoxetamine, montelukast, vilaprisan, Losartan).

Midazolam also known. 8-chloro-6-(2-fluorophenyl)-1-methyl-4H-imidazo[1,5-a][1,4] is a diazepine of the formula (II):

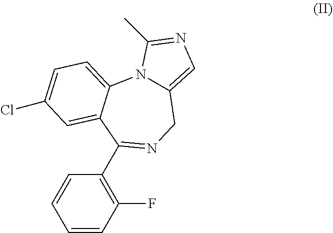 [SMILES: Cc1ncc2n1-c1ccc(Cl)cc1C(c1ccccc1F)=NC2]

Midazolam is a well-documented product with sedative, anxiolytic, amnesic and hypnotic properties. It is commercially available in the form of its hydrochloride, for example in the form of a glycerine-based syrup sold for example under the trade name VERSED®, which contains 2.5 mg/ml of midazolam. It is also sold in the form of its maleate salt, for example in tablets containing 7.5 mg or 15 mg per tablet for example under the trade mark DORMICUM®. For example, a product which is formulated for administration via the buccal route is EPISTATUS®. Buccal formulations of midazolam are also disclosed in EP1323422. Midazolam is a short-acting benzodiazepine. It is exclusively metabolized by CYP3A.

The terms “standard prescribed dosage”, “normal prescribed dosage”, “usual dosage” and “standard dosage” can be used interchangeably and refer to the standard and authorized prescribed drug dosage according to the leaflet instruction. The standard dose may vary depending on the form or the route the drug is being administered. For example the “standard dosage” for midazolam sold in ampoules forms of 1 ml (5 mg midazolam), 3 ml (15 mg midazolam), 5 ml (5 mg midazolam) and 10 ml (50 mg midazolam) for iv, im (intramuscular) and rectal use can be found in Table 1. In another example the “standard” dosage for midazolam sold as a tablet in the form of 7.5 mg and 15 mg tablets, the standard dose is 7.5 to 15 mg for adults, where 7.5 mg is the usual dose for e.g. older patients.

TABLE 1
Standard dosage of Midazolam ampoules

| | | Adults ≥60 years/ | |
|---|---|---|---|
| | | in critical condition | Children and |
| Indication | Adults <60 years | or at risk | adolescents |
| Sedation while | iv | iv | iv 6 months - 5 |
| preserving | Initial dose: 2-2.5 mg | Initial dose: | years: |
| consciousness | Titration dose: 1 mg | 0.5-1 mg | Initial dose: 0.05-0.1 |
| | Total dose: 3.5-7.5 mg | Titration dose: | mg/kg body weight |
| | | 0.5-1 mg | (bw) Total dose: |
| | | Total dose: | ≤6 mg |
| | | ≤3.5 mg | iv 6-12 years: |
| | | | Initial dose: 0.025- |
| | | | 0.05 mg/kg |
| | | | bw; Total dose: |
| | | | ≤10 mg |
| | | | iv 13-16 years: |
| | | | like adults |
| | | | rectal >6 months: |
| | | | 0.3-0.5 mg/kg bw |
| | | | im 1-15 years: |
| | | | 0.05-0.15 mg/kg |
| | | | body weight |
| Premedication | iv | iv | rectal >6 months: |
| before induction of | 1-2 mg, repeated | Initial dose: 0.5 mg | 0.3-0.5 mg/kg bw |
| anesthesia | | Slow incremental | |
| | | increase as needed | |
| Indication | Adults <60 years | Adults ≥60 years/ | Children and |
| | | in critical condition | adolescents |
| | | or at risk | |
| | im | im | im 1-15 years: |
| | 0.07-0.1 mg/kg body weight | 0.025-0.05 mg/kg | 0.08-0.2 mg/kg |
| | | body weight | body weight |
| Anesthetic | iv | iv | not indicated in |
| introduction | 0.2 mg/kg body weight | 0.05-0.15 mg/kg | pediatrics |
| | (0.2-0.35 mg/kg body weight | bw | |
| | without premedication) | (0.2 mg/kg bw | |
| | | without | |
| | | premedication) | |
| Sedating | iv | iv | not indicated in |
| component in | intermittent doses of 0.03-0.1 | Lower doses than | pediatrics |
| combination | mg/kg body weight or | recommended for | |
| anesthesia | continuous intravenous | adults <60 years | |
| | infusion of 0.03-0.1 mg/kg/h | | |
| Sedation in | iv | | iv Pregnancy age |
| intensive care | initial dose of 0.03-0.3 mg/ | | ≤32 weeks: |
| | kg body weight with gradual | | 0.03 mg/kg bw/h |
| | increase of 1-2.5 mg | | iv Pregnancy >32 |
| | maintenance dose: 0.03-0.2 | | weeks to 6 months: |
| | mg /kg/h | | 0.06 mg/kg bw/h |
| Indication | Adults <60 years | Adults ≥60 years/ | Children and |
| | | in critical condition | adolescents |
| | | or at risk | |
| | | | iv Age >6 months: |
| | | | Initial dose: 0.05-0.2 |
| | | | mg/kg |
| | | | bw Maintenance |
| | | | dose: 0.06-0.12 mg/ |
| | | | kg bw/h |

The term “therapeutically effective amount,” as used herein, refers to an amount of a compound sufficient to treat, ameliorate, or prevent the identified disease or condition, or to exhibit a detectable therapeutic, prophylactic, or inhibitory effect. The effect can be detected by, for example, an improvement in clinical condition, or reduction in symptoms. The precise effective amount for a subject will depend upon the subject's body weight, size, and health; the nature and extent of the condition; and the therapeutic or combination of therapeutics selected for administration. Where a drug has been approved by the U.S. Food and Drug Administration (FDA), a “therapeutically effective amount” refers to the dosage approved by the FDA or its counterpart foreign agency for treatment of the identified disease or condition.

As used herein, a patient “in need of risdiplam therapy” is a patient who would benefit from administration of risdiplam. The patient may be suffering from any disease or condition for which risdiplam therapy may be useful in ameliorating symptoms. Risdiplam is being developed for treating spinal muscular atrophy.

As used herein, a patient in need of “midazolam therapy” is understood to be a patient in need of sedative therapy, therapy of sleep disturbances or seizures.

In any of the embodiments described herein, including but not limited to providing risdiplam for use in treatment of SMA, the use of risdiplam in the manufacture of a medicament for treatment of SMA, and treatment methods involving the advice, warnings, discontinuation, reducing dosing or dose titration downwards, the packages and kits, and/or the methods of preparing or packaging risdiplam, the risdiplam, uses, methods, packages, kits, advice, warnings, discontinuation or dose titration may apply to any drug that is a substrate of CYP3A enzymes. The embodiments apply to any other drug that is a substrate of CYP3A enzymes. In yet a particular embodiment the dosage of CYP3A substrates should get reduced versus the normal prescribed dosage of the CYP3A substrate. In another particular embodiment the dosage of CYP3A substrates is reduced by 10%, 15%, 20%, 30%, 40%, 50%, 60%, 70%, 80%, 90%, or 98% of the standard prescribed dosage of the CYP3A substrate.

In one aspect, the invention relates to avoiding concomitant use of risdiplam in patients with any CYP3A substrates, in particular midazolam at any dose. It is understood that the patient is in need of risdiplam therapy and in need of treatment with a CYP3A substrate, in particular in need of a sedative therapy such as provided by midazolam. In such methods, the CYP3A substrate, in particular midazolam, is avoided during risdiplam administration, or vice versa. In related methods, the CYP3A substrate, in particular midazolam, is discontinued during risdiplam administration.

In embodiments of such methods, the methods avoid concomitant administration of risdiplam and the CYP3A substrate, in particular midazolam, at equivalent doses by other routes. For instance, based on Table 1 intravenous (i.v) dosing of midazolam 3.5 to 7.5 mg i.v per day or in the form of tablet from 7.5 to 15 mg per day orally.

In another embodiment, concomitant administration of midazolam at any dose should be avoided during risdiplam therapy due to the potential for reduced clearance of midazolam. The midazolam dose that is avoided may be within a dosage range (for example and without limitation, between 10% to 100% of the standard dosage of midazolam, between 30% to 100% of the standard dosage of midazolam, or between 40% to 100% of the standard dosage of midazolam).

In examples of methods involving avoiding midazolam standard dosage, the methods comprise administering a therapeutically effective amount of risdiplam to the patient, and administering an alternative sedative therapy that is not midazolam and preferably is not a substrate of CYP3A.

In some embodiments, the patient is administered midazolam at an alternative dosage (i.e., at a lower dose than the standard dosage). Thus, in various embodiments, the patient is administered midazolam at a dose that is 10%, 15%, 20%, 25%, 30%, 35%, 40%, 45%, 50%, 55%, 60%, 65%, 70%, 75%, 80%, 85%, 90%, 95% or 98% of the standard dosage per day.

In some aspects, the disclosure provides a method of administering risdiplam therapy to a patient in need of risdiplam therapy (e.g., a patient with SMA), involving administering to the patient a therapeutically effective amount of risdiplam, and advising the patient in any one, two, three or more of the following ways:
a) advising the patient that midazolam at any dose, in particular at standard dosage, should be avoided or discontinued,
b) advising the patient that co-administration of risdiplam with midazolam at standard dosage can alter the therapeutic effect or adverse reaction profile of midazolam,
c) advising the patient that co-administration of risdiplam and midazolam at standard dosage results in an increase in exposure to midazolam, and/or
d) advising the patient that midazolam at any dose should be used with caution in patients receiving risdiplam due to the potential for reduced midazolam clearance and/or increased midazolam exposure.

In various embodiments of the methods described herein, a method of administering risdiplam and midazolam concurrently is provided wherein the patient is administered risdiplam at 0.2 mg/kg for patients between 2 months and 2 years, at 0.25 mg/kg for patients above age 2 years and with a body weight of less than 20 kg and at 5 mg for patients with a body weight of more than or equal to 20 kg, and the patient is administered a reduced dosage of midazolam, given orally or by other routes (reduced relative to a patient not taking risdiplam, or relative to the previously administered midazolam dosage in the patient). For instance the dosage of midazolam is decreased by 10%, 15%, 20%, 25%, 30%, 35%, 40%, 45%, 50%, 55%, 60%, 65%, 70%, 75%, 80%, 85%, 90%, 95% or 98%, particularly by 10%, 15%, 20%, 25% or 30%, more particularly by 10% or 15%, of the standard dosage of midazolam.

In some embodiments, the dose of midazolam is reduced by 10%, 15%, 20%, 25%, 30%, 35%, 40%, 45%, 50%, 55%, 60%, 65%, 70%, 75%, 80%, 85%, 90%, 95% or 98% of the standard dosage of midazolam. In specific embodiments, the dose of midazolam is reduced by about 40%, 45%, 50%, 55%, 60%, 65%, 70%, 75%, 80%, 85%, 90%, 95% or 98% relative to the previously administered dose. In further embodiments, the dose of midazolam is reduced by about 10%, 15%, 20%, 25%, 30%, 35%, 40%, 45%, 50%, 55%, 60%, 65%, 70%, 75%, 80%, 85%, 90%, or more relative to the previously administered midazolam dose, or to a dose ranging from about 50% to about 98%, or about 60% to about 90% of the previously administered dose.

In more specific embodiments, the dose of midazolam is reduced by 10%, 15%, 20%, 25% or 30% more particularly by 10% or 15% of the standard dosage of midazolam

As noted above, in any of the embodiments described herein, including but not limited to discontinuation or dose reduction, the packages and kits, and/or the methods of preparing or packaging risdiplam, the risdiplam, uses, methods, packages, kits, advice, warnings, discontinuation or dose titration may apply not only to oral standard dosage ofmidazolam (i.e 7.5 mg), but also to any other equivalent dose given by another route e.g. intravenous (i.v.) dosing of midazolam.

In another aspect, a package or kit is provided comprising risdiplam, optionally in a container, and a package insert, package label, instructions or other labeling including instructions or directions for any of the methods disclosed herein.

The package insert, package label, instructions or other labeling may further comprise directions for treating a patient in need of risdiplam, e.g. with SMA by administering risdiplam, e.g., at a dosage 0.2 mg/kg for patient between 2 months and 2 years, or at a dosage of 0.25 mg/kg for patient older than 2 years and with a body weight of less than 20 kg, and at 5 mg for patient with a body weight of more than or equal to 20 kg.

In a related aspect, the disclosure provides a method of preparing or packaging a risdiplam medicament comprising packaging risdiplam, optionally in a container, together with a package insert or package label or instructions for any of the methods disclosed herein.

In some embodiments, a method of treating a patient in need of risdiplam is disclosed comprising providing, selling or delivering any of the kits of disclosed herein to a hospital, physician or patient.

In some embodiments, a method of treating a patient in need of midazolam at reduced dosage of 10%, 15%, 20%, 25%, 30%, 35%, 40%, 45%, 50%, 55%, 60%, 65%, 70%, 75%, 80%, 85%, 90%, 95% or 98%, particularly by 10%, 15%, 20%, 25% or 30%, more particularly by 10% or 15%, of the standard dosage of midazolam is provided comprising providing or delivering a kit comprising midazolam together with a package insert or package label or instructions for any of the methods disclosed herein, to a hospital, physician or patient.

According to the here within described invention more particular embodiments of the invention are described below:

### Embodiment 1

Risdiplam for use in treating a patient in need of risdiplam therapy wherein the dosage of CYP3A substrate for administration to a patient is reduced by 10%, 15%, 20%, 25%, 30%, 35%, 40%, 45%, 50%, 55%, 60%, 65%, 70%, 75%, 80%, 85%, 90%, 95% or 98% of the standard dosage of CYP3A substrate during concomitant administration of risdiplam.

### Embodiment 2

Risdiplam for use in the treatment of SMA, wherein the dosage of CYP3A substrate for administration to a patient is reduced by 10%, 15%, 20%, 25%, 30%, 35%, 40%, 45%, 50%, 55%, 60%, 65%, 70%, 75%, 80%, 85%, 90%, 95% or 98% of the standard dosage of CYP3A substrate during concomitant administration of risdiplam.

### Embodiment 3

Risdiplam for use in treating a patient in need of risdiplam therapy wherein the normal prescribed dosage of CYP3A substrate for administration to a patient is reduced by 10%, 15%, 20%, 25%, 30%, 35%, 40%, 45%, 50%, 55% 60%, 65%, 70%, 75%, 80%, 85%, 90%, 95% or 98% of the standard dosage of CYP3A substrate during concomitant administration of risdiplam.

### Embodiment 4

Risdiplam for use in the treatment of SMA, wherein the normal prescribed dosage of CYP3A substrate administration to a patient is reduced by 10%, 15%, 20%, 25%, 30%, 35%, 40%, 45%, 50%, 55%, 60%, 65%, 70%, 75%, 80%, 85%, 90%, 95% or 98% of the standard dosage of CYP3A substrate during concomitant administration of risdiplam.

### Embodiment 5

Risdiplam for use in treating a patient in need of risdiplam therapy wherein the oral or i.v standard dosage of midazolam for administration to a patient is reduced by 10%, 15%, 20%, 25%, 30%, 35%, 40%, 45%, 50%, 55% 60%, 65%, 70%, 75%, 80%, 85%, 90%, 95% or 98% of the standard dosage of midazolam during concomitant administration of risdiplam.

### Embodiment 6

Risdiplam for use in the treatment of SMA, wherein the standard dosage of midazolam oral dose or i.v dose for administration to a patient wherein midazolam is reduced by 10%, 15%, 20%, 25%, 30%, 35%, 40%, 45%, 50%, 55% 60%, 65%, 70%, 75%, 80%, 85%, 90%, 95% or 98% of the standard dosage of midazolam during concomitant administration of risdiplam.

### Embodiment 7

Risdiplam for use in treating a patient in need of risdiplam therapy and of a CYP3A substrate therapy, wherein the dosage of CYP3A substrate for administration to a patient is reduced by 10%, 15%, 20%, 25%, 30%, 35%, 40%, 45%, 50%, 55%, 60%, 65%, 70%, 75%, 80%, 85%, 90%, 95% or 98% of the standard dosage of CYP3A substrate during concomitant administration of risdiplam.

### Embodiment 8

Risdiplam for use in treating a patient in need of risdiplam therapy and of a CYP3A substrate therapy wherein the normal prescribed dosage of CYP3A substrate for administration to a patient is reduced by 10%, 15%, 20%, 25%, 30%, 35%, 40%, 45%, 50%, 55%, 60%, 65%, 70%, 75%, 80%, 85%, 90%, 95% or 98% of the standard dosage of CYP3A substrate during concomitant administration of risdiplam.

### Embodiment 9

Risdiplam for use in treating a patient in need of risdiplam therapy and a of midazolam therapy wherein the oral, i.v, im, rectal, buccal or any other route of administration's standard dosage of midazolam for administration to a patient is reduced by 10%, 15%, 20%, 25%, 30%, 35%, 40%, 45%, 50%, 55%, 60%, 65%, 70%, 75%, 80%, 85%, 90%, 95% or 98% of the standard dosage of midazolam during concomitant administration of risdiplam.

### Embodiment 10

Risdiplam for use in treating a patient in need of risdiplam therapy wherein the standard dosage of midazolam oral dose ori.v dose, for administration to a patient is reduced by 10%, 15%, 20%, 25%, 30%, 35%, 40%, 45%, 50%, 55%, 60%, 65%, 70%, 75%, 80%, 85%, 90%, 95% or 98% of the standard dosage of midazolam during concomitant administration of risdiplam.

### Embodiment 11

Risdiplam for use in the treatment of SMA, wherein the standard dosage of midazolam oral or i.v. dose for administration to a patient wherein midazolam is reduced by 10%, 15%, 20%, 25%, 30%, 35%, 40%, 45%, 50%, 55% 60%, 65%, 70%, 75%, 80%, 85%, 90%, 95% or 98% of the standard dosage of midazolam during concomitant administration of risdiplam.

### Embodiment 12

Risdiplam for use in treating a patient in need of risdiplam therapy wherein the risdiplam is for administering to the patient at a therapeutically effective amount, and avoiding concomitant administration of midazolam at any oral dose or at any intravenous (i.v.) dose.

### Embodiment 13

The risdiplam for use according to any one of the embodiments 1 to 12, wherein the midazolam dosage is reduced by 10%, 15%, 20%, 25%, 30%, 35%, 40%, 45%, 50%, 55%, 60%, 65%, 70%, 75%, 80%, 85%, 90%, 95% or 98% of the standard dosage of midazolam during risdiplam and midazolam concomitant administration.

### Embodiment 14

The risdiplam for use according to any one of embodiments 1 to 13 wherein the total daily dose of risdiplam is administered to the patient at 0.2 mg/kg for patients between 2 months and 2 years, at 0.25 mg/kg for patients older than 2 years and with a body weight of less than 20 kg, and at 5 mg for patients with a body weight of more than or equal to 20 kg.

### Embodiment 15

The risdiplam for use according to any one of embodiments 1 to 12 wherein the total daily dose of risdiplam is administered orally 0.2 mg/kg for patients between 2 months and 2 years, at 0.25 mg/kg for patients older than 2 years and with a body weight of less than 20 kg, and at 5 mg for patients with a body weight of more than or equal to 20 kg.

### Embodiment 16

The risdiplam for use according to any one of embodiments 1 to 15 wherein the total daily dose of risdiplam is administered orally, a dose of 0.2 mg/kg for patients between 2 months and 2 years.

### Embodiment 17

The risdiplam for use according to any one of embodiments 1 to 16 wherein the total daily dose of risdiplam is administered orally a dose of 0.25 mg per kilogram of body weight of risdiplam per day for a patient older than 2 years and with a body weight of less than 20 kg.

### Embodiment 18

The risdiplam for use according to any one of embodiments 1 to 16 wherein the total daily dose of risdiplam is administered orally a dose 5 mg of risdiplam per day for a patient with a body weight of more than or equal to 20 kg.

### Embodiment 19

The risdiplam for use according to any one of embodiments 1 to 18 wherein the midazolam is administered orally in unit dosage forms that are capsules or tablets.

### Embodiment 20

The risdiplam for use according to any one of embodiments 1 to 16 wherein the amount of midazolam in the unit dosage form is 7.5 mg or 15 mg.

### Embodiment 21

The risdiplam for use according to any one of embodiments 1 to 20 wherein during concomitant midazolam administration, 0.20 mg per kilogram of body weight of risdiplam per day for a patient between 2 months and 2 years is administered to the patient.

### Embodiment 22

The risdiplam for use according to any one of embodiments 1 to 20 wherein during concomitant midazolam administration, 0.25 mg per kilogram of body weight of risdiplam per day for a patient older than 2 years and with a body weight of less than 20 kg is administered to the patient.

### Embodiment 23

The risdiplam for use according to any one of embodiments 1 to 20 wherein during concomitant midazolam administration, 5 mg of risdiplam per day for a patient with a body weight of more than or equal to 20 kg is administered to the patient.

### Embodiment 24

The risdiplam for use according to any one of embodiments 1 to 20 wherein during concomitant midazolam administration, the risdiplam is administered at a total daily dosage of 0.20 mg per kilogram of body weight of risdiplam per day for a patient between 2 months and 2 years.

### Embodiment 25

The risdiplam for use according to any one of embodiments 1 to 20 wherein during concomitant midazolam administration, the risdiplam is administered at a total daily dosage of 0.25 mg per kilogram of body weight of risdiplam per day for a patient older than 2 years and with a body weight of less than 20 kg.

### Embodiment 26

The risdiplam for use according to any one of embodiments 1 to 20 wherein during concomitant midazolam administration, the risdiplam is administered at a total daily dosage of 5 mg of risdiplam per day for a patient with a body weight of more than or equal to 20 kg.

### Embodiment 27

The risdiplam for use according to any one of embodiments 1 to 20 wherein during concomitant midazolam administration, the risdiplam is administered at a total daily dosage of about 0.20 mg per kilogram of body weight of risdiplam per day for a patient between 2 months and 2 years.

### Embodiment 28

The risdiplam for use according to any one of embodiments 1 to 20 wherein during concomitant midazolam administration the risdiplam is administered at a total daily dosage of about 0.25 mg per kilogram of body weight of risdiplam per day for a patient older than 2 years and with a body weight of less than 20 kg.

### Embodiment 29

The risdiplam for use according to any one of embodiments 1 to 20 wherein during concomitant midazolam administration the risdiplam is administered at a total daily dosage of about 5 mg of risdiplam per day for a patient with a body weight of more than or equal to 20 kg.

### Embodiment 30

The risdiplam for use according to any one of embodiments 1 to 29 wherein the patient has SMA.

### Embodiment 31

The risdiplam for use according to any one of embodiments 1 to 30 wherein the patient has a type I SMA; type II SMA or type III SMA.

### Embodiment 32

The risdiplam for use according to any one of embodiments 1 to 31 wherein the patient has a type II SMA or type III SMA.

### Embodiment 33

The risdiplam for use according to any one of embodiments 1 to 31 wherein the patient has a type I SMA.

### Embodiment 34

The risdiplam for use according to any one of embodiments 1 to 31 wherein the patient has a type II SMA.

### Embodiment 35

The risdiplam for use according to any one of embodiments 1 to 31 wherein the patient has a type III SMA.

### Embodiment 36

Midazolam for use in treating a patient in need of midazolam therapy, for example, with a need of sedative therapy, or for treatment of sleep disturbance or seizures, during concomitant administration of risdiplam, wherein the standard dosage of midazolam for administration to the patient is reduced by 10%, 15%, 20%, 25%, 30%, 35%, 40%, 45%, 50%, 55%, 60%, 65%, 70%, 75%, 80%, 85%, 90%, 95% or 98%.

### Embodiment 37

Midazolam for use in the treatment of seizures during concomitant administration of risdiplam, wherein the standard dosage of midazolam for administration to the patient is reduced.

### Embodiment 38

Midazolam for use in the treatment of seizures wherein the midazolam is for administration at an oral dose or at an intravenous (i.v.) dose wherein midazolam is avoided during concomitant administration of risdiplam.

### Embodiment 39

Midazolam for use in treating a patient in need of midazolam therapy wherein the midazolam is for administration at an oral dose or at an intravenous (i.v.) dose wherein midazolam is avoided during concomitant administration of risdiplam.

### Embodiment 40

The midazolam for use according to any one of the embodiments 36 to 39 wherein the midazolam dosage is reduced by 10%, 15%, 20%, 25%, 30%, 35%, 40%, 45%, 50%, 55%, 60%, 65%, 70%, 75%, 80%, 85%, 90%, 95% or 98% of the standard dosage of midazolam during risdiplam administration.

### Embodiment 41

The midazolam for use according to any one of the embodiments 36 to 40 wherein the midazolam is avoided to avoid the potential for a reduced clearance of midazolam or the potential for an increased exposure to midazolam.

### Embodiment 42

The midazolam for use according to any one of the embodiments 36 to 41 wherein during concomitant midazolam administration, 0.20 mg per kilogram of body weight of risdiplam per day for a patient between 2 months and 2 years, 0.25 mg per kilogram of body weight of risdiplam per day for a patient older than 2 years and with a body weight of less than 20 kg, or 5 mg of risdiplam per day for a patient with a body weight of more than or equal to 20 kg of risdiplam is administered to the patient.

### Embodiment 43

The midazolam for use according to any one of the embodiments 36 to 42 wherein during concomitant midazolam administration the risdiplam is administered at a total daily dosage of 0.20 mg per kilogram of body weight of risdiplam per day for a patient between 2 months and 2 years, 0.25 mg per kilogram of body weight of risdiplam per day for a patient older than 2 years and with a body weight of less than 20 kg or 5 mg of risdiplam per day for a patient with a body weight of more than or equal to 20 kg.

### Embodiment 44

The midazolam for use according to any one of the embodiments 36 to 42 wherein during concomitant midazolam administration the risdiplam is administered at a total daily dosage of about 0.25 mg per kilogram of body weight of risdiplam per day for a patient older than 2 years and with a body weight of less than 20 kg, or about 5 mg of risdiplam per day for a patient with a body weight of more than or equal to 20 kg.

### Embodiment 45

The midazolam for use according to any one of the embodiments 36 to 44 wherein the patient has SMA.

### Embodiment 46

The midazolam for use according to any one of the embodiments 36 to 45 wherein the patient has a has a type I SMA, a type II SMA or type III SMA.

### Embodiment 47

The midazolam for use according to any one of the embodiments 36 to 46 wherein the patient has a has a type II SMA or type III SMA.

### Embodiment 48

The midazolam for use according to any one of the embodiments 36 to 46 wherein the patient has a has a type I SMA.

### Embodiment 49

The midazolam for use according to any one of the embodiments 36 to 46 wherein the patient has a has a type II SMA.

### Embodiment 50

The midazolam for use according to any one of the embodiments 36 to 46 wherein the patient has a has a type III SMA.

### Embodiment 51

Use of midazolam at a total daily dose that is reduced by 10%, 15%, 20%, 25%, 30%, 35%, 40%, 45%, 50%, 55%, 60%, 65%, 70%, 75%, 80%, 85%, 90%, 95% or 98% of the standard dosage of midazolam, during concomitant use of risdiplam at a dose of about 0.20 mg per kilogram of body weight of risdiplam per day for a patient between 2 months and 2 years, about 0.25 mg per kilogram of body weight of risdiplam per day for a patient older than 2 years and with a body weight of less than 20 kg, or about 5 mg of risdiplam per day for a patient with a body weight of more than or equal to 20 kg.

### Embodiment 52

Use of midazolam at a total daily dose that is reduced by 10%, 15%, 20%, 25%, 30%, 35%, 40%, 45%, 50%, 55%, 60%, 65%, 70%, 75%, 80%, 85%, 90%, 95% or 98% of the standard dosage of midazolam, during concomitant use of risdiplam at a dose of 0.20 mg per kilogram of body weight of risdiplam per day for a patient between 2 months and 2 years, 0.25 mg per kilogram of body weight of risdiplam per day for a patient older than 2 years and with a body weight of less than 20 kg, or 5 mg of risdiplam per day for a patient with a body weight of more than or equal to 20 kg.

### Embodiment 53

Midazolam for use at a total daily dose that is reduced, by 10%, 15%, 20%, 25%, 30%, 35%, 40%, 45%, 50%, 55%, 60%, 65%, 70%, 75%, 80%, 85%, 90%, 95% or 98% of the standard dosage of midazolam during concomitant use of risdiplam at a dose of 0.20 mg per kilogram of body weight of risdiplam per day for a patient between 2 months and 2 years, about 0.25 mg per kilogram of body weight of risdiplam per day for a patient older than 2 years and with a body weight of less than 20 kg, or about 5 mg of risdiplam per day for a patient with a body weight of more than or equal to 20 kg.

### Embodiment 54

Midazolam for use at a total daily dose that is reduced, by 10%, 15%, 20%, 25%, 30%, 35%, 40%, 45%, 50%, 55%, 60%, 65%, 70%, 75%, 80%, 85%, 90%, 95% or 98% of the standard dosage of midazolam during concomitant use of risdiplam at a daily dose of 0.25 mg per kilogram of body weight of risdiplam per day for a patient older than 2 years and with a body weight of less than 20 kg, or 5 mg of risdiplam per day for a patient with a body weight of more than or equal to 20 kg.

### Embodiment 55

The use of midazolam or midazolam for use of any one of embodiments 51 to 54 for avoiding potential for a reduced clearance of midazolam or potential for an increased exposure to midazolam.

### Embodiment 56

The use of midazolam or midazolam for use of any one of embodiments 51 to 55 wherein the midazolam is in one or more unit dosage forms that are capsules or tablets.

### Embodiment 57

The use of midazolam or midazolam for use of any one of embodiments 51 to 56 wherein the amount of midazolam in each of the one or more unit dosage forms is 7.5 mg or 15 mg.

### Embodiment 58

The use of midazolam or midazolam for use of any one of embodiments 51 to 57 in a patient that has SMA.

### Embodiment 59

Use of risdiplam at a total daily dose of 0.25 mg per kilogram of body weight of risdiplam per day for a patient older than 2 years and with a body weight of less than 20 kg, or 5 mg of risdiplam per day for a patient with a body weight of more than or equal to 20 kg, for the treatment of SMA in a patient concomitantly receiving a reduced dose of midazolam of 10%, 15%, 20%, 25%, 30%, 35%, 40%, 45%, 50%, 55%, 60%, 65%, 70%, 75%, 80%, 85%, 90%, 95% or 98% of the standard dosage of midazolam.

### Embodiment 60

Risdiplam for use at a total daily dose of 0.25 mg per kilogram of body weight of risdiplam per day for a patient older than 2 years and with a body weight of less than 20 kg, or 5 mg of risdiplam per day for a patient with a body weight of more than or equal to 20 kg for the treatment of SMA in a patient concomitantly receiving a reduced dose of midazolam of 10%, 15%, 20%, 25%, 30%, 35%, 40%, 45%, 50%, 55%, 60%, 65%, 70%, 75%, 80%, 85%, 90%, 95% or 98% of the standard dosage of midazolam.

### Embodiment 61

Use of risdiplam or risdiplam for use according to any one of embodiments 59-60 wherein SMA is selected from the group consisting of type I SMA, Type II SMA or type III SMA.

### Embodiment 62

A pharmaceutical composition comprising a pharmaceutically acceptable excipient and 5 mg of risdiplam for use to treat SMA in a patient concomitantly receiving a reduced dose of midazolam of 10%, 15%, 20%, 25%, 30%, 35%, 40%, 45%, 50%, 55%, 60%, 65%, 70%, 75%, 80%, 85%, 90%, 95% or 98% of the standard dosage of midazolam.

### Embodiment 63

A package or kit comprising (a) risdiplam, optionally in a container, and (b) a package insert, package label, instructions or other labeling for the use or risdiplam for use according to any of embodiments 51 to 62.

### Embodiment 64

The pharmaceutical composition according to embodiment 62, wherein the pharmaceutical composition comprises risdiplam formulated as oral aqueous solution by dissolving the risdiplam in a buffer system at pH of less than pH 4, particularly less than pH 3.8, more particularly less than pH 3.6, most particularly pH 3.0 to 3.2, in order to provide sufficiently high drug concentration, e.g. citric buffer system, malate buffer system, maleate buffer system, or tartrate buffer system, most particularly tartrate buffer system.

### Embodiment 65

The pharmaceutical composition according to embodiment 62, wherein the pharmaceutical composition comprises risdiplam as a dry powder or granulation for constitution of an oral solution.

### Embodiment 66

The pharmaceutical composition according to embodiment 62, wherein the pharmaceutical composition comprises risdiplam, a diluent, such as sorbitol, isomalt, or particularly mannitol, and combinations thereof, which ensure fast dissolution of the powder blend during constitution of the oral solution.

### Embodiment 67

The pharmaceutical composition according to any one of embodiments 62 to 66, wherein the pharmaceutical composition comprises:
risdiplam; and
a buffer system selected from citrate, malate, maleate or tartrate, particularly malate or tartrate, most particularly tartrate; or alternatively the corresponding acid of a buffer system alone as acidifier, particularly tartaric acid.

### Embodiment 68

The pharmaceutical composition according to any one of embodiments 62 to 67, wherein the pharmaceutical composition comprises:
7-(4,7-diazaspiro[2.5]octan-7-yl)-2-(2,8-dimethylimidazo[1,2-b]pyridazin-6-yl)pyrido[1,2-a]pyrimidin-4-one or a pharmaceutically acceptable salt thereof;
a buffer system, particularly a buffer system selected from citrate, malate, maleate or tartrate, more particularly malate or tartrate, most particularly tartrate; or alternatively the corresponding acid of a buffer system alone as acidifier, particularly tartaric acid; and
a diluent, particularly mannitol or a mixture of mannitol and isomalt, more particularly mannitol.

### Embodiment 69

The pharmaceutical composition according to any one of embodiments 62 to 68, wherein the pharmaceutical composition comprises:
1 to 10% wt of 7-(4,7-diazaspiro[2.5]octan-7-yl)-2-(2,8-dimethylimidazo[1,2-b]pyridazin-6-yl)pyrido[1,2-a]pyrimidin-4-one or a pharmaceutically acceptable salt thereof,
5 to 15% wt of a buffer system, particularly a buffer system selected from citrate, malate, maleate or tartrate, more particularly malate or tartrate, most particularly tartrate; or alternatively the corresponding acid of a buffer system alone as acidifier, particularly tartaric acid;
to 70% wt of a diluent, particularly mannitol or a mixture of mannitol and isomalt, more particularly mannitol;
1 to 4% wt of an antioxidant, particularly ascorbic acid;
0.5 to 2% wt of a stabilizer, particularly disodium edetate;
0.5 to 2% w of a lubricant, particularly PEG6000;
0 to 3% wt of a sweetener, particularly sucralose or sodium saccharin, most particularly sucralose; and
0 to 20% wt of a flavor, particularly strawberry flavor or vanilla flavor; wherein the total amount of ingredients does not exceed 100% wt.

### Embodiment 70

The pharmaceutical composition according to any one of embodiments 62 to 69, wherein the pharmaceutical composition comprises:
2 to 6% wt of 7-(4,7-diazaspiro[2.5]octan-7-yl)-2-(2,8-dimethylimidazo[1,2-b]pyridazin-6-yl)pyrido[1,2-a]pyrimidin-4-one or a pharmaceutically acceptable salt thereof,
9 to 13% wt of a tartrate buffer system;
to 55% wt of a mannitol as first diluent and 8 to 10% wt of isomalt as second diluent;
1 to 3% wt of ascorbic acid as antioxidant;
0.5 to 2% wt of disodium edetate as stabilizer;
0.5 to 2% w of PEG6000 as lubricant;
1.5 to 2% wt of sucralose as sweetener; and
13 to 17% wt of strawberry flavor;

wherein the total amount of ingredients does not exceed 100% wt.

The following example is intended merely to illustrate the practice of the present invention and is not provided by way of limitation.

In the present application, the following abbreviations and definitions are used:

| | |
|---|---|
| AE | Adverse event |
| ALT | Alanine aminotransferase |
| AR_{Auc} | Accumulation ratio for AUC |
| ARCₘₐₓ | Accumulation ratio for Cmax |
| AST | Aspartate aminotransferase |
| AUC | Area under the plasma concentration-time curve |
| AUC_{inf} | Area under the plasma concentration-time curve |
| | extrapolated to infinity |
| AUCₗₐₛₜ | Area under the plasma concentration-time curve from |
| | time 0 to the time of last quantifiable concentration |
| AUCₜₐᵤ | Area under the plasma concentration-time curve over a |
| | dosing interval |
| BMI | Body mass index |
| CLₛₛ/F | Apparent total plasma clearance at steady state |
| Cₘₐₓ | Maximum observed plasma concentration |
| CNS | Central nervous system |
| CRF | Case Report Form |
| C_{trough} | Trough observed plasma concentration |
| CYP | Cytochrome P450 |
| DDI | Drug-drug interaction |
| ECG | Electrocardiogram |
| eCRF | Electronic Case Report Form |
| FDA | Food and Drug Administration |
| FSH | Follicle-stimulating hormone |
| GI | Gastrointestinal |
| HBsAg | Hepatitis B surface antigen |
| HBcAb | Hepatitis B core antibody |
| HIV | Human immunodeficiency virus |
| IB | Investigator’s Brochure |
| NADP+ | Nicotinamide adenine dinucleotide phosphate |
| NADPH | Nicotinamide Adenine Dinucleotide Phosphate Hydrogen |
| OTC | Over-the-counter |
| PK | Pharmacokinetic(s) |
| PR | Pulse rate |
| QD | Once daily |
| QRS | QRS complex |
| QT | QT interval |
| QTc | QT corrected for heart rate |
| QTcF | QT corrected for heart rate using Fridericia's formula |
| RBC | Red blood cell |
| SAE | Serious adverse event |
| SMA | Spinal muscular atrophy |
| SMN | Survival motor neuron |
| SoA | Schedule of Activities |
| t½ | Apparent plasma terminal elimination half-life |
| Tₘₐₓ | Time of maximum observed plasma concentration |
| TSH | Thyroid-stimulating hormone |
| ULN | Upper limit of normal |
| US | United States |
| WBC | White blood cell |

### Example 1

Evaluation of inhibition of cytochromes P450 catalytic activities in human liver microsomes by risdiplam.

The purpose of this study was to determine the direct and time-dependent inhibition of cytochrome P450 (CYP) isoforms CYP1A2, CYP2B6, CYP2C8, CYP2C9, CYP2C19, CYP2D6, and CYP3A4/5 by risdiplam in a human liver microsomal test system.

No significant reversible or time-dependent inhibition of CYPs 1A2, 2B6, 2C8, 2C9, 2C19, or 2D6 was detected with risdiplam test concentrations of up to 12.5 μM.

Risdiplam demonstrated low potential to cause direct inhibition of CYP3A4/5 (midazolam 1′-hydroxylase and testosterone 6p-hydroxylase). Risdiplam tested up to 12.5 μM exhibited a maximal 28% and 55% inhibition for midazolam and testosterone as a substrate, respectively. An IC50 of 11 μM was estimated for risdiplam inhibition of testosterone metabolism. (In this study a testosterone concentration of 50 μM was used.).

Results when risdiplam was pre-incubated with HLM indicated the potential for risdiplam to be a time-dependent inhibitor of CYP3A4/5. The KI value and kinact values were estimated as 13 μM and 0.065 min-1, respectively. However, there is some uncertainty in these values because the time-dependent inactivation effect did not reach saturation with tested risdiplam concentrations due to solubility limitation.

The direct and time-dependent inhibition data for risdiplam are summarized in Table 2. The CYP3A4 KI and kinact parameters are summarized in Table 3.

Positive control inhibitors for direct and time-dependent inhibition determinations demonstrated a properly functioning test system.

TABLE 2
Direct and Time-Dependent Inhibition of Cytochrome P450
Enzymes by risdiplam

| | | Direct Inhibition | | | Time-dependent |
|---|---|---|---|---|---|
| | | risdiplam | | | Inhibition |
| P450 | | | % Control | | Fold Change |
| Isoform | Substrate | IC50 | activity at | | in IC50* |
| CYP1A2 | Phenacetin | >12.5 | 94 | 101 | NA |
| CYP2B6 | Bupropion | >12.5 | 88 | 94 | NA |
| CYP2C8 | Amodiaquine | >12.5 | 93 | 91 | NA |
| CYP2C9 | Diclofenac | >12.5 | 83 | 82 | NA |
| CYP2C19 | (S)- | >12.5 | 102 | 97 | NA |
| | Mephenytoin | | | | |
| CYP2D6 | Dextro- | >12.5 | 97 | 95 | NA |
| | methorphan | | | | |
| CYP3A4 | Midazolam | >12.5 | 72 | 72 | >2.6 |
| CYP3A4 | Testosterone | 11 | 44 | 46 | >3.0 |

NA-not applicable
*The fold change in IC50 was not applicable for time-dependent inhibition
evaluation for all enzymes. This is because there was insufficient inhibition for
IC50 values to be calculated.

TABLE 3
CYP3A4 inactivation parameters for risdiplam

| Probe Substrate | kinact (min⁻¹) | K_{I} (μM) |
|---|---|---|
| Midazolam | 0.065 | 13 |

### Objective

The purpose of this study was to determine whether risdiplam inhibits human cytochrome P450 (CYP) catalytic activities in vitro using model substrates and human liver microsomes.

### Regulatory Compliance

This was a non-GLP study conducted in accordance with the Study Protocol and the applicable Corning Life Sciences—Discovery Labware Standard Operating Procedures (SOPs).

### Major Computer Systems Assistance:

The major computer/software systems used in this study included Microsoft EXCEL, and Analyst® software v1.6.2 (Applied Biosystems) for generating LC-MS/MS data.

### Test Articles

Test article risdiplam was supplied by the Sponsor. Information about the test article is described below in (Table 4).

TABLE 4
Test Article Information

| Test Article Designation: | Risdiplam |
|---|---|
| Lot Purity: | 99.92% |
| Molecular Weight (free acid/base): | 401.464 |
| Physical State Provided (solid or solution): | Solid |
| Stock Solution Solvent: | Ethanol:water (80%:20%) |
| Storage Conditions (solid): | −20° C. |
| Storage Conditions (Stock Solutions): | Prepare fresh |

Inhibition of cytochrome P450 enzyme catalytic activity is a major mechanism of metabolism-based drug interactions. Determination of IC50 shift or KI/kinact values (for time- and NADPH-dependent inhibition) aids in the prediction of metabolism-based drug-drug interactions⁽¹⁻³⁾.

### Test System Description

This study was carried out using Corning UltraPool HLM 150™ pooled human liver microsomes (Corning catalog no. 452117). The batch data sheet for the HLM preparation used is found in Table 5. Table 5Table 5: Batch Data sheet
All cytochrome P450 assays conducted at 0.8 mg/ml protein (except CYP3A4 which was at 0.5 mg/ml) with an NADPH generating system (1.3 mM NADP, 3.3 mM glucose 6-phosphate and 0.4 U/ml glucose 6-phosphate dehydrogenase), 3.3 mM MgCl2, and incubated for 20 minutes or 10 minutes (CYP2C8, CYP2C9, CYP3A4 and CYP4A). 0.1 M Potassium phosphate buffer (pH 7.4) was used for all P450 enzymes except CYP2B6, CYP2C8, and CYP2C19 (0.05 M) and CYP2A6, CYP2C9, and CYP4A which used 0.1 M Tris (pH 7.5). The FMO assay was conducted in the same volume and protein concentration in 0.05 M glycine buffer (pH 9.5) with the same NADPH generating system, 3.3 mM MgCl2, 1.2 mM diethylenetriaminepentacetic acid, 0.5 mg/ml Triton X-100 and incubated for 10 minutes. UGT Glucuronidation assays contained 0.5 mg/ml protein for UGT1A1 and 1A4, 0.1 mg/ml for 1A6, 0.15 mg/ml for 1A9 and 0.8 mg/ml for 2B7, along with 2 mM UDPGA, 10 mM MgCl2, 25 ug/ml Alamethicin in 50 mM Tris-HCl buffer (pH 7.5). UGT1A1 was incubated for 30 minutes, 1A4 for 20 minutes, 1A6 for 15 minutes, 1A9 for 10 minutes and 2B7 for 25 minutes. Activities expressed as pmol product per (mg protein×minute) except cytochrome c reductase which is expressed as nmol product per (mg protein×minute).
The Western Blot assay was carried out using standard protocols. SDS-gel electrophoresis was by the method of Laemmli (Laemmli, U. K, 1970, Nature, 227: 680-685.). CYP protein abundance in HLM was quantitated using authentic standards derived from recombinant P450 isoforms.
The pool is comprised of equal milligrams of microsome per donor.
HAZARD WARNING: This microsome preparation was prepared from freshly frozen human tissues. All donor tissues have tested negative for pathogens by PCR for the following: HIV I/II, HTLV I/II, CMV, HBV and HCV, however, we recommend that this material be considered a potential biohazard.
Donors with positive serology for CMV are identified in the donor demographic sheet with a single asterisk. Donors with CMV serology unknown are identified with a double asterisk. Donors CMV negative for serology are unm

### Experimental: CYP450 Inhibition

### Study Design:

Enzyme/Substrate Pairs and Incubation Conditions

IC50 and IC50 shift assays were conducted to evaluate direct and time-dependent enzyme inhibition by the test article. Enzyme/substrate pairs and incubation conditions are listed in Table 6 and Table 7. The final organic solvent concentration in the incubations was constant for all concentrations of the test article.

Direct Inhibition Assay

Reaction mixtures (400 μL) contained seven non-zero concentrations of test article (0, 0.1, 0.2, 0.5, 1.3, 3.2, 8.0 and 12.5 μM), microsomal protein, an NADPH-regenerating system (1.3 mM NADP+, 3.3 mM glucose-6-phosphate, 0.4 U/mL glucose-6-phosphate dehydrogenase, 3.3 mM magnesium chloride) and one concentration of a probe substrate (Table 6) in 100 mM potassium phosphate buffer (pH 7.4). Reactions were initiated by addition of diluted HLM protein and incubated at 37° C. for the times indicated (Table 6). Reactions were stopped by addition of 100 μL of the stop solution (0.1% formic acid in acetonitrile containing a stable-isotope labeled internal standard) and placement on ice.

TABLE 6
Enzyme/Substrate Pairs (Direct Inhibition)

| | | Substrate | HLM | Incubation |
|---|---|---|---|---|
| P450 | | Concentration | Concentration | Time |
| Isoform | Substrate | IC50 (μM) | (mg/mL) | (min) |
| CYP1A2 | Phenacetin | 40 | 0.2 | 10 |
| CYP2B6 | Bupropion | 80 | 0.1 | 5 |
| CYP2C8 | Amodiaquine | 1.5 | 0.02 | 5 |
| CYP2C9 | Diclofenac | 5 | 0.05 | 5 |
| CYP2C19 | (S)-Mephenytoin | 40 | 0.3 | 10 |
| CYP2D6 | Dextromethorphan | 5 | 0.1 | 5 |
| CYP3A4 | Midazolam | 3 | 0.02 | 5 |
| CYP3A4 | Testosterone | 50 | 0.05 | 10 |

Time Dependent Inhibition Assay (IC₅₀ Shift)

Pre-incubation reaction mixtures contained seven non-zero concentrations of test article (0, 0.1, 0.2, 0.5, 1.3, 3.2, 8.0 and 12.5 μM) and microsomal protein, with and without an NADPH-regenerating system (1.3 mM NADP+, 3.3 mM glucose-6-phosphate, 0.4 U/mL glucose-6-phosphate dehydrogenase, and 3.3 mM magnesium chloride) in 100 mM potassium phosphate buffer (pH 7.4). Incubations without an NADPH-regenerating system had water in its place. Reactions were initiated by HLM and incubated at 37° C.

After 30 min of pre-incubation time, a 40 μL (or 80 μL for CYP2C19 only) aliquot was transferred into a pre-warmed secondary reaction mixture (400 μL final volume) containing the NADPH-regenerating system and one concentration of probe substrate (Table 7) in 100 mM potassium phosphate buffer (pH 7.4). Reactions were incubated at 37° C. for the times specified in Table 7 and stopped by addition of 100 μL of the stop solution (0.1% formic acid in acetonitrile containing a stable-isotope labeled internal standard) and placement on ice.

TABLE 7
Enzyme/Substrate Pairs (Time-Dependent Inhibition, IC50 Shift)

| | | | | Pre- | |
|---|---|---|---|---|---|
| | | Sub- | | Incu- | Incu- |
| | | strate | HLM | bation | bation |
| P450 | | Conc. | Conc.¹ | Time | Time |
| Isoform | Substrate | (μM) | (mg/mL) | (min) | (min) |
| CYP1A2 | Phenacetin | 40 | 2.0 | 30 | 10 |
| CYP2B6 | Bupropion | 80 | 1.0 | 30 | 5 |
| CYP2C8 | Amodiaquine | 1.5 | 0.2 | 30 | 5 |
| CYP2C9 | Diclofenac | 5 | 0.5 | 30 | 5 |
| CYP2C19 | (5)-Mephenytoin | 40 | 1.5 | 30 | 10 |
| CYP2D6 | Dextromethorphan | 5 | 1.0 | 30 | 5 |
| CYP3A4 | Midazolam | 3 | 0.2 | 30 | 5 |
| CYP3A4 | Testosterone | 50 | 0.5 | 30 | 10 |

¹HLM concentrations are for the pre-incubation, and the secondary HLM concentrations are the same as in Table 6.

Time Dependent Inhibition (CYP3A4 K_{I} AND K_{inact})

Pre-incubation reaction mixtures contained eight non-zero concentrations of test article (0, 0.1, 0.2, 0.5, 1.3, 3.2, 8.0 and 12.5 μM), microsomal protein, and an NADPH-regenerating system (1.3 mM NADP+, 3.3 mM glucose-6-phosphate, 0.4 U/mL glucose-6-phosphate dehydrogenase, and 3.3 mM magnesium chloride) in 100 mM potassium phosphate buffer (pH 7.4). Reactions were initiated by addition of diluted HLM protein and incubated at 37° C. After six different pre-incubation times (Table 8) a 40 μL aliquot of the secondary incubation was transferred into a pre-warmed secondary reaction mixture (400 μL final volume) containing the NADPH-regenerating system and midazolam as probe substrate in 100 mM potassium phosphate buffer (pH 7.4). Reactions were incubated at 37° C. and stopped by combining an aliquot of the reaction mix (200 μL) with stop solution (50 μL, 0.1% formic acid in acetonitrile containing a stable-isotope labelled internal standard) and placement on ice.

TABLE 8
Enzyme/Substrate Pairs (Time-Dependent Inhibition,
CYP3A4-K_{I} and k_{inact})

| | | Substrate | HLM | Pre- | |
|---|---|---|---|---|---|
| P450 | | Conc. | Conc.¹ | Incubation | Incubation |
| Isoform | Substrate | (μM) | (mg/mL) | Time (min) | Time (min) |
| CYP3A4 | Midazolam | 15 | 0.2 | 0, 4, 15, | 5 |
| | | | | 30, 45 | |
| | | | | and 60 | |

¹HLM concentrations are for the pre-incubation, and the secondary HLM concentrations are the same as in Table 6.

### Replicates

All incubations were conducted in duplicate.

### Analytical

The samples were centrifuged to compress the precipitated protein into a pellet. The supernatant was stored at −20° C. for subsequent analysis by LC/MS/MS. The probe substrate metabolites were analyzed by LC/MS/MS (Table 9)³. Catalytic activities were calculated using standard curves for each metabolite based on peak area ratios (analyte/internal standard).

TABLE 9
LC/MS/MS Methods for Analysis of P450 Probe Substrate Metabolites

| | | | Internal standard |
|---|---|---|---|
| P450 | | Metabolite | (MRM mass |
| Isoform | Substrate | (MR1VI mass transition) | transition) |
| CYP1A2 | Phenacetin | Acetamidophenol | Acetamidophenol-[¹³C2¹⁵N] |
| | | (152→110) | (155→111) |
| CYP2B6 | Bupropion | OH-Bupropion | OH-Bupropion-[D6](262→244) |
| | | (256→139) | |
| CYP2C8 | Amodiaquine | N-Desethylamodiaquine | N-Desethylamodiaquine-[D3] |
| | | (330→285) | (333→285) |
| CYP2C9 | Diclofenac | 4′-OH Diclofenac | 4′-OH Diclofenac-[¹³C6] |
| | | (312→268) | (316→272) |
| CYP2C19 | (S)-Mephenytoin | 4′-OH S-Mephenytoin | 4′-OH S-Mephenytoin-[D3] |
| | | 235→150) | (238→150) |
| CYP2D6 | Dextromethorphan | Dextrorphan (258→157) | Dextrorphan-[D3](261→157) |
| CYP3A4 | Midazolam | 1′-OH Midazolam | 1′-OH Midazolam-[¹³C3] |
| | | (342→203) | (347→208) |
| CYP3A4 | Testosterone | 6β-OH Testosterone | 6β-OH Testosterone-[D7] |
| | | (305→269) | (312→276) |

### Positive Controls

The following positive control CYP inhibitors were used for IC50 (Table 10) and IC50 shift assays (Table 11) in accordance with the methods described above using a 30 min pre-incubation time point with and without NADPH for IC50 shift assays.

For the K_{I}/kinact assay, the positive control time-dependent inhibitor was included at a single concentration (0.8 μM) using the same pre-incubation time points as the test article.

TABLE 10
Positive Control Inhibitors for Direct Inhibition, Acceptance Criteria
and Results Obtained

| | Direct Inhibition | Acceptable range | Results obtained |
|---|---|---|---|
| P450 Isoform | Positive Control | IC50 value (uM) | |
| CYP1A2 | 7,8-Benzoflavone | 0.0010-0.070 | 0.010 |
| CYP2B6 | Ketoconazole | 0.45-15 | 3.4 |
| CYP2C8 | Montelukast | 0.0070-0.20 | 0.044 |
| CYP2C9 | Sulfaphenazole | 0.15-1.5 | 0.34 |
| CYP2C19 | S-Benzylnirvanol | 0.10-1.5 | 0.21 |
| CYP2D6 | Quinidine | 0.020-0.20 | 0.046 |
| CYP3A4/ | Ketoconazole | 0.0030-0.15 | 0.018 |
| Midazolam | | | |
| CYP3A4/ | Ketoconazole | 0.0050-0.090 | 0.014 |
| Testosterone | | | |

TABLE 11
Positive Control Inhibitors for Time-dependent Inhibition,
Acceptance Criteria and Results Obtained

| | Time-dependent Inhibition | | |
|---|---|---|---|
| | | Acceptable | Results |
| | | range | obtained |
| P450 Isoform | Positive Control | IC50 value¹ (μM) | |
| CYP1A2 | Furafylline | 0.0035-0.085 | 0.020 |
| CYP2B6 | Ticlopidine | 0.030-0.20 | 0.053 |
| CYP2C8 | Gemfibrozil | 0.080-9.5 | 0.76 |
| | glucuronide | | |
| CYP2C9 | Tienilic acid | 0.025-0.15 | 0.045 |
| CYP2C19 | S-Fluoxetine | 0.60-15 | 2.1 |
| CYP2D6 | Paroxetine | 0.015-0.20 | 0.042 |
| CYP3A4/ | Azamulin | 0.0010-0.015 | 0.0048 |
| Midazolam | | | |
| CYP3A4/ | Azamulin | 0.0035-0.040 | 0.011 |
| Testosterone | | | |

¹IC50 value after a 30 min pre-incubation calculated based on inhibitor concentrations in the secondary incubation.

### Calculations

### Percent Remaining Activity

% remaining=(C_{+I}/C^{-I})*100

Where:

C_{+I} concentration of probe substrate metabolite formed in presence of inhibitor

C_{-I} concentration of probe substrate metabolite formed in absence of inhibitor

### IC₅₀

IC₅₀ values were determined by non-linear regression using XLfit (model 205, a four parameter logistic fit); the maximum and minimum values were fixed at 100% and 0%.

Fit=A+((B−A)/(1+((C/x)\hat{ }D)))

### Where:

A is the minimum y value

B is the maximum y value

C is IC₅₀ and is the inhibitor concentration associated with 50% inhibition

D is the slope factor

### IC₅₀ Shift

IC₅₀ shift=IC₅₀(−NADPH)/IC₅₀(+NADPH)

### Where:

IC₅₀ (−NADPH) is the IC₅₀ value obtained after pre-incubation in absence of NADPH

IC₅₀ (+NADPH) is the IC₅₀ value obtained after pre-incubation in presence of NADPH

K_{I} and k_{inact}

To determine k_{inact} and K_{I} values, the natural logarithm of the residual activity (corrected for any loss of activity observed over time in absence of inhibitor) was plotted against the pre-incubation time for each test article concentration. The first order rate constant for inactivation (λ or k_{abs}) was estimated from slopes of the linear portion of the curves. Inactivation kinetic parameters (k_{inact} and K_{I}) were determined using non-linear regression using GraphPad Prism software version 6.01 as:

$$λ = \frac{{K}_{inacz .} \cdot [ I ]}{{K}_{I} + [ I ]} \qquad$$

### Where,

λ or k_{obs} is the first order rate constant for inactivation estimated from the slope of plot of LN(residual activity) vs. pre-incubation time
[I] is the inhibitor concentration
k_{inact} is the maximal rate of enzyme inactivation [min]
K_{I} is the concentration of inhibitor resulting in 50% of the maximum enzyme inactivation

Because the time-dependent inactivation effect did not reach saturation with risdiplam concentrations due to the limitation of test article solubility, the kinact/K_{I} ratio was estimated based on the slope of linear fitting of kobs versus test concentrations (y=slope·x).

### Results and Discussion:

### Direct Inhibition of CYP Isoforms

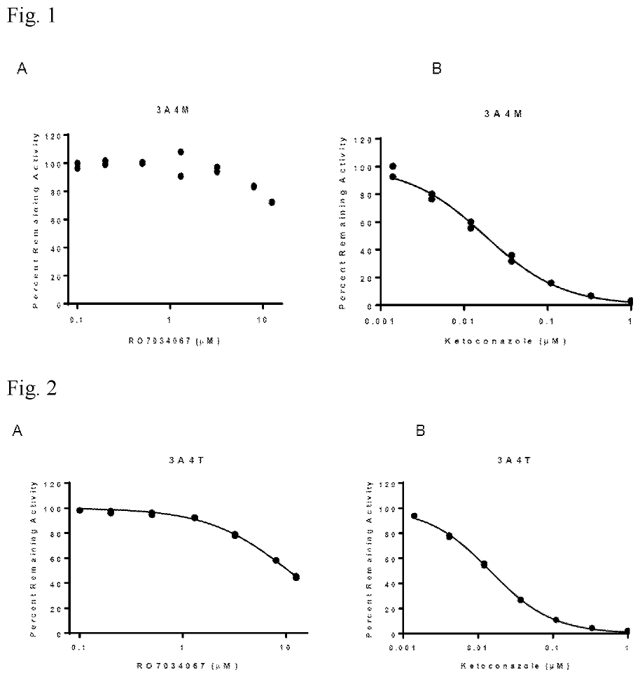

The results for direct inhibition of CYP isoforms by risdiplam are summarized in Table 2. No significant reversible inhibition of CYPs 1A2, 2B6, 2C8, 2C9, 2C19, or 2D6 was detected with risdiplam test concentrations of up to 12.5 μM. Risdiplam demonstrated low potential to cause direct inhibition of CYP3A4/5 where a maximal 28% and 55% inhibition was observed for midazolam and testosterone as substrates, respectively. An IC50 of 11 μM was estimated for inhibition of CYP3A4/5-mediated testosterone 6β-hydroxylation. Here a substrate concentration of 50 μM was employed, below the Kₘ concentration of 65 μM. Individual CYP3A4 inhibition data with risdiplam are listed in Table 12 and Table 13. The percent activity remaining as a function of risdiplam or positive control concentration is presented graphically in FIG. 1 and FIG. 2. All positive control inhibitors met the acceptance criteria (see Table 10), thus demonstrating a properly functioning test system.

TABLE 12
Effect of risdiplam on CYP3A4-Midazolam Activity

| risdiplam (μM) | % Remaining | | IC50 (μM) |
|---|---|---|---|
| 12.5 | 72 | 72 | <12.5 |
| 8.0 | 84 | 83 | |
| 3.2 | 94 | 97 | |
| 1.3 | 108 | 91 | |
| 0.50 | 101 | 100 | |
| 0.20 | 99 | 102 | |
| 0.10 | 100 | 96 | |

TABLE 13
Effect of risdiplam on CYP3A4-Testosterone Activity

| risdiplam (μM) | % Remaining | | IC50 (μM) |
|---|---|---|---|
| 12.5 | 44 | 46 | 11 |
| 8.0 | 58 | 58 | |
| 3.2 | 78 | 79 | |
| 1.3 | 93 | 92 | |
| 0.50 | 96 | 95 | |
| 0.20 | 98 | 96 | |
| 0.10 | 98 | 98 | |

### Time-Dependent Inhibition of CYP Isoforms

The results for time-dependent inhibition of CYP isoforms by risdiplam are summarized in Table 2 No significant time-dependent inhibition of CYPs 1A2, 2B6, 2C8, 2C9, 2C19, or 2D6 was detected with risdiplam test concentrations of up to 12.5 μM. An increase in the apparent potency of risdiplam in midazolam and testosterone hydroxylase inhibition was observed when risdiplam was pre-incubated with HLM in the presence of NADPH with IC50 shift >2. These results indicated the potential for risdiplam to be a time-dependent inhibitor of CYP3A4/5.

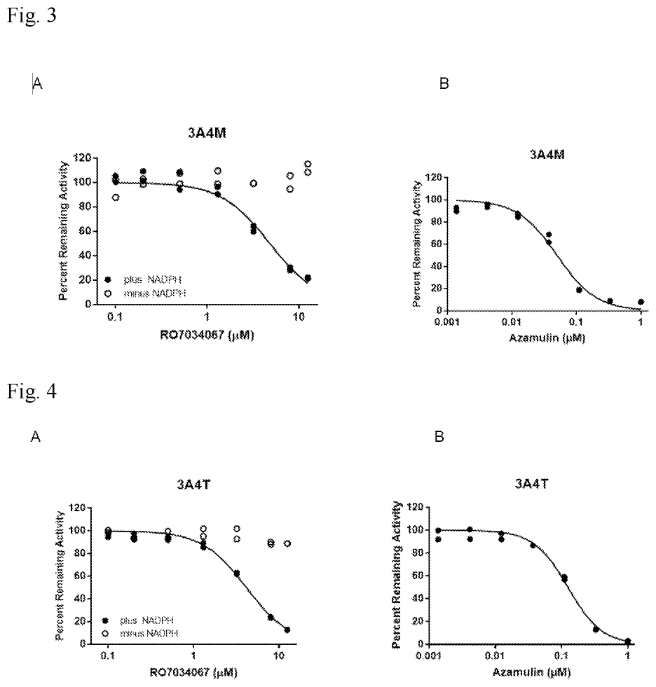

The individual time-dependent inhibition data for CYP3A4 with risdiplam and the positive controls are listed in Table 14, Table 15, Table 16 and Table 17._The data are presented graphically in FIG. 3 and FIG. 4. All positive control inhibitors met the acceptance criteria (see Table 11), thus demonstrating a properly functioning test system.

TABLE 14
Effect of risdiplam on CYP3A4-Midazolam
Inactivation (plus NADPH)

| risdiplam - pre-incubation | | | |
|---|---|---|---|
| (μM) | % Remaining | | IC₅₀ (μM) |
| 12.5 | 22 | 21 | 4.8 |
| 8.0 | 28 | 30 | |
| 3.2 | 60 | 64 | |
| 1.3 | 90 | 96 | |
| 0.50 | 94 | 109 | |
| 0.20 | 101 | 109 | |
| 0.10 | 101 | 105 | |

TABLE 15
Effect of risdiplam on CYP3A4-Midazolam
Inactivation (minus NADPH)

| risdiplam - pre-incubation | | | |
|---|---|---|---|
| (μM) | % Remaining | | IC₅₀ (μM) |
| 12.5 | 115 | 108 | >12.5 |
| 8.0 | 95 | 106 | |
| 3.2 | 99 | 100 | |
| 1.3 | 99 | 110 | |
| 0.50 | 99 | 108 | |
| 0.20 | 99 | 103 | |
| 0.10 | 88 | 103 | |

TABLE 16
Effect of risdiplam on CYP3A4-Testosterone
Inactivation (plus NADPH)

| risdiplam - pre-incubation | | | |
|---|---|---|---|
| (μM) | % Remaining | | IC₅₀ (μM) |
| 12.5 | 13 | 13 | 4.1 |
| 8.0 | 23 | 24 | |
| 3.2 | 63 | 62 | |
| 1.3 | 90 | 85 | |
| 0.50 | 94 | 92 | |
| 0.20 | 98 | 93 | |
| 0.10 | 97 | 95 | |

TABLE 17
Effect of risdiplam on CYP3A4-Testosterone Inactivation (minus NADPH)

| risdiplam-pre-incubation | | | |
|---|---|---|---|
| (μM) | % Remaining | | IC50 (μM) |
| 12.5 | 89 | 89 | >12.5 |
| 8.0 | 90 | 88 | |
| 3.2 | 93 | 102 | |
| 1.3 | 102 | 95 | |
| 0.50 | 100 | 93 | |
| 0.20 | 94 | 93 | |
| 0.10 | 101 | 99 | |

K_{I} and k_{inact} Determination

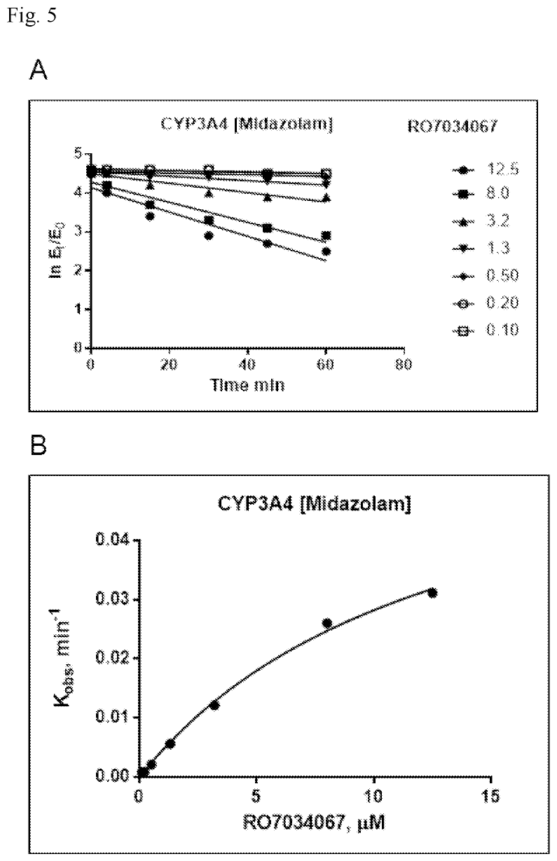

The results for K_{I} and k_{inact} determination for CYP 3A4/5 by risdiplam are summarized in Table 3. The K_{I} value and k_{inact} values were estimated as 13 μM and 0.065 min⁻¹, respectively. However, there is some uncertainty in these values because the time-dependent inactivation effect did not reach saturation with risdiplam concentrations tested due to solubility limitations. The individual percent activity remaining data with risdiplam and the positive control is listed in Table 18. The inactivation plots and the kobs versus concentration plots are shown in FIG. 5.

TABLE 18
Percent CYP3A4 activity remaining with midazolam as substrate

| Pre- | | | | | | | | | Positive |
|---|---|---|---|---|---|---|---|---|---|
| incubation | risdiplam μM | | | | | | | | control |
| time (min) | 12.5 | 8.0 | 3.2 | 1.3 | 0.50 | 0.20 | 0.10 | 0 | Azamulin |
| 60 | 10 | 15 | 36 | 48 | 59 | 65 | 65 | 71 | 11 |
| 45 | 13 | 19 | 39 | 55 | 68 | 72 | 72 | 78 | 12 |
| 30 | 17 | 25 | 49 | 65 | 75 | 78 | 78 | 81 | 15 |
| 15 | 29 | 41 | 62 | 71 | 77 | 80 | 80 | 86 | 19 |
| 4 | 62 | 76 | 89 | 82 | 87 | 90 | 90 | 96 | 30 |
| 0 | 100 | 100 | 100 | 100 | 100 | 100 | 100 | 100 | 100 |

### Conclusions

In conclusion, risdiplam demonstrated low potential to cause direct or time-dependent inhibition of CYP1A2, CYP2B6, CYP2C8, CYP2C9, CYP2C19 and CYP2D6.

Risdiplam showed some inhibition of CYP3A4/5 at the concentrations tested. In addition, risdiplam demonstrated low potential to cause time-dependent inhibition of all isoforms, except for CYP3A4/5. A follow-up CYP3A4/5 inactivation kinetics assay resulted in a K_{I} and k_{inact} estimates of 13 μM and 0.065 min, respectively.

### Example 2

A Phase I, 2-part, open label study is being carried out to investigate the safety, tolerability and pharmacokinetics of multiple doses of risdiplam and the effect of risdiplam on the pharmacokinetics of midazolam following oral administration in healthy participants.

Part 1 of this study investigates the safety, tolerability, and pharmacokinetics (PK) of multiple oral doses of risdiplam administered once daily (QD) for 14 days to healthy participants. The PK and safety data collected in Part 1 will be used to define the dose and to enable the start of Part 2 of this study.

Part 2 of this study assesses the effect of multiple oral doses of risdiplam on the PK of midazolam following administration to healthy participants, to check for drug-drug interaction of risdiplam with cytochrome P450 3A substrates.

### Study Design

This will be a Phase I, 2-part, open-label, non-randomized study to investigate the safety, tolerability, and PK of multiple doses of risdiplam (Part 1) and the effect of risdiplam on the PK of midazolam (Part 2) following oral administration in healthy adult male and female participants.

### Treatment Groups and Duration

| | | |
|---|---|---|
| Study Treatment Name: | Risdiplam (RO7034067) | Midazolam |
| | | (Part 2 only) |
| Dose Formulation: | Powder for constitution | Solution |
| | to an oral solution | |
| Dose: | 5 mg (6.66 mL) (Part 1) | 2 mg (1 mL) |
| Route of administration: | Oral | Oral |

Note:
Part 2 will be defined based on Part 1 data.

### Length of Study

The total duration of the study for each participant will be up to approximately 8 weeks divided as follows:
Screening: Up to 27 days (Days −28 to −2).
In clinic period: Day −1 to Day 16 (Part 1) or Day −1 to Day 18 (Part 2).
Non-residential visits: Days 18 and 20 (Part 1) or Days 20 and 22 (Part 2).
Safety Follow-up (Post-study): 10±2 days post final dose of study drug in Parts 1 and 2.

### End of Study

The end of the study is defined as the date when the last participant last visit occurs.

### Participant Population

The participants in this study will be healthy female and male volunteers between 18 and 55 years of age, inclusive, who fulfill all of the given eligibility criteria.

### Inclusion/Exclusion Criteria

### Inclusion Criteria

Participants are eligible to be included in the study only if all of the following criteria apply:
1) Willingness and ability to provide written consent to participate in the clinical trial.
2) Healthy participants.
   Healthy status is defined by the Investigator based on detailed review of medical and surgical history, results of physical examination, vital signs, 12-lead ECG, and laboratory assessments (hematology, coagulation, blood chemistry, serology, and urinalysis).
3) Male and female participants aged 18 to 55 years of age, inclusive, at Screening. Female participants: A female participant is eligible to participate if she is a woman of non-childbearing potential (WONCBP).
4) A body mass index (BMI) of 18.0 to 32.0 kg/m2, inclusive, at Screening
5) Use of adequate contraception methods during the treatment period and until 4 months after last study drug administration. Males must refrain from donating sperm during this same period.
   Contraception methods for male participants considered as acceptable for the study:
      With non-pregnant female partners, use contraceptive measures such as a condom with spermicide plus an additional contraceptive method that together result in a failure rate of <1% per year, with partners who are women of childbearing potential. The additional contraceptive method must be 1 of the following: diaphragm in combination with spermicide, intrauterine device, injectable or implantable contraceptives, oral hormonal contraceptives (e.g., “progesterone only pills,” tablets, patch, or vaginal ring with both estrogen and progesterone). Contraception is required during the treatment period and for at least 4 months after the last dose of risdiplam.
      With pregnant female partners, use contraceptive measures such as a condom to avoid exposing the embryo during the treatment period and for at least 28 days after the last dose of risdiplam.
      Abstinence (including those who practice abstinence as part of their normal and preferred lifestyle, periodic abstinence, e.g., calendar, ovulation, symptothermal, or post ovulation methods) and withdrawal are not acceptable methods of contraception in this study. Note that only WONCBP and men are eligible for the study.
6) Willingness and ability to complete all aspects of the study.

### Exclusion Criteria

Participants are excluded from the study if any of the following criteria apply:
1) History of any clinically significant GI, renal, hepatic, broncho-pulmonary, neurological, psychiatric, cardiovascular, endocrinological, hematological, or allergic disease, metabolic disorder, cancer, or cirrhosis.
2) Concomitant disease or condition that could interfere with, or treatment of which might interfere with, the conduct of the study, or that would, in the opinion of the Investigator, pose an unacceptable risk to the participant in this study, including but not limited to the following:
   Any major illness within 1 month before Screening or any febrile illness within 1 week prior to Screening and up to first study drug administration.
3) History or evidence of any medical condition potentially altering the absorption, metabolism, or elimination of drugs.
4) Surgical history of the GI tract affecting gastric motility or altering the GI tract (with the exception of uncomplicated appendectomy and hernia repair) (a cholecystectomy is exclusionary).
5) History or presence of clinically significant ECG abnormalities (based on the average of 3 consecutive measurements [if the first measurement is out of range, complete 2 more and take the average]) (e.g., PQ/PR interval >210 ms, QT interval corrected for heart rate using Fridericia's formula [QTcF]>450 ms for males and QTcF>470 ms for females) or cardiovascular disease (e.g., cardiac insufficiency, coronary artery disease, cardiomyopathy, congestive heart failure, family history of congenital long QT syndrome, family history of sudden death).
6) History of malignancy in the past 5 years.
7) Confirmed (based on the average of 3 consecutive measurements [if the first measurement is out of range, complete 2 more and take the average]) systolic blood pressure >140 or <90 mmHg, and diastolic blood pressure >90 or <50 mmHg at Screening only.
8) Confirmed (based on the average of 3 consecutive measurements) resting pulse rate (PR) >100 or <40 bmp at Screening only.
9) Clinically significant abnormalities (as judged by the Investigator) in laboratory test results (including hematology, chemistry panel, and urinalysis). In case of uncertain or questionable results, tests performed during Screening may be repeated on Day −1 to confirm eligibility.
10) Positive result on human immunodeficiency virus (HIV)-1, HIV-2, hepatitis B virus, or hepatitis C virus (serology) tests at Screening.
11) Any suspicion or history of alcohol abuse and/or any history or suspicion of regular consumption/addiction of drugs of abuse within 2 years prior to study drug administration or a positive drug screen test as performed at Screening.
12) Any consumption of tobacco-containing products (including but not limited to the following: smoking cigarettes, cigars, etc.) from 1 month before Screening until Follow-up.
13) Donation of blood or blood products for transfusion over 500 mL within 3 months prior to first study drug administration and for the duration of the study.
14) Participation in an investigational drug medicinal product or medical device study within 90 days prior to Screening.
15) Use of prohibited medications or herbal remedies.
16) Any clinically significant history of hypersensitivity or allergic reactions, either spontaneous or following study drug administration, or exposure to food or environmental agents.
17) History of hypersensitivity to any of the excipients in the formulation of the study drug.
18) History of hypersensitivity to midazolam or any other benzodiazepine or its formulation ingredients (this applies to participants in Part 2 only).
19) For Part 2 participants: history of acute angle glaucoma.
20) Participants who, in the Investigator's judgment, pose a suicidal risk, or any participant with a history of suicidal or homicidal attempts.
21) Participants under judicial supervision, guardianship, or curatorship.
22) Participants who, in the opinion of the Investigator, should not participate in this study.

### Number of Participants

In total a maximum of 40 participants may be enrolled in this study as follows:
Part 1: 8 participants will be enrolled in order to obtain 6 evaluable participants.
Part 2: 28 participants will be enrolled in order to obtain at least 26 evaluable participants.

The additional 4 participants are in case the dropout rate in Part 2 is higher than expected in order to achieve 26 evaluable participants.

In Part 1, participants will receive a dose of 5 mg risdiplam QD for 14 consecutive days. The dose of 5 mg risdiplam has been shown to be safe and well tolerated for more than 1 year of treatment in patients with SMA. The decision to proceed to Part 2 of the study will be made following review of all available safety and tolerability data, including AEs, ECGs, vital signs, laboratory safety test results (i.e., hematology, clinical chemistry, and urinalysis) collected up to (and including) 48 hours after last study drug administration and available plasma PK data up to (and including) 24 hours after last study drug administration from a minimum of 4 Part 1 participants. The risdiplam dose in Part 2 will be determined based on the PK and safety data obtained in Part 1, with the aim to achieve an average exposure (mean AUC over a dosing interval [AUCtau] at steady state) of 2000 ng·h/mL in Part 2 (i.e., the therapeutic exposure observed in SMA patients).

A Dose Escalation Meeting will be conducted prior to the start of Part 2, in order to evaluate the Part 1 data and to select the risdiplam dose to be administered in Part 2 of this study.

In Part 2, all study participants will receive a single oral dose of 2 mg midazolam on Day 1. On Day 3, the 14-day QD treatment period with risdiplam will begin (targeting a mean AUCtau at steady state of 2000 ng·h/mL; the precise dose will be based on the results of Part 1), with single dose administration of 2 mg midazolam again on Day 15 (1 hour after the thirteenth dose of risdiplam).

In both study parts, PK blood samples will be collected at timepoints specified in Table 19. Safety monitoring will be performed throughout the study as described later.

The Schedule of Activities (SoA) for Parts 1 and 2 is provided in Table 19.

### Concomitant Medications

No concomitant medication is permitted, except acetaminophen, hormone replacement therapy for post-menopausal women, and medication to treat AEs.

Any medication or vaccine (including over-the-counter [OTC] or prescription medicines, approved dietary and herbal supplements, nutritional supplements) and any non-medication interventions (e.g., individual psychotherapy, cognitive behavioral therapy, smoking cessation therapy, and rehabilitative therapy) used by a participant from 30 days prior to Screening until the Follow-up visit must be recorded along with reason for use, dates of administration (including start and end dates), and dosage information (including dose and frequency).

### Permitted Therapy

Participants who use hormone replacement therapy should continue their use. Acetaminophen, at doses of ≤2 g/day, is permitted for use as needed. Other concomitant medication required to treat AEs may be considered on a case-by-case basis by the Investigator.

### Prohibited Therapy

All medications (prescription and OTC) taken within 30 days of Screening will be recorded on the appropriate eCRF.

As a general rule, no concomitant medication will be permitted, with the exception of acetaminophen, hormone replacement therapy for post-menopausal women, and medications to treat AEs, unless the rationale for exception is discussed and clearly documented between the Investigator and the Sponsor and archived in the site file.

Participants must abstain from taking prescription or non-prescription drugs (including vitamins and dietary or herbal supplements) within 14 days or 5 half-lives (whichever is longer) before the start of study treatment until completion of the Follow-up visit, unless, in the opinion of the Investigator and Sponsor, the medication will not interfere with the study.

The following medications are explicitly prohibited:
Any inhibitor of CYP3A4 (e.g., ketoconazole, miconazole, itraconazole, fluconazole, erythromycin, clarithromycin, ranitidine, cimetidine).
Any inducer of CYP3A4 (e.g., rifampicin, rifabutin, glucocorticoids, carbamazepine, phenytoin, phenobarbital, St. John's wort).
Any organic cation transporter 2 and MATE substrates (e.g., amantadine, cimetidine, memantine, amiloride, famotidine, metformin, pindolol, ranitidine, procainamide, varenicline, acycolovir, ganciclovir, oxaliplatin, cephalexin, cephradine, fexofenadine).
Medications with known or potential retinal toxicity (e.g., chloroquine and hydroxychloroquine, thioridazine, retigabin, vigabatrin, desferoxamine, topiramate, latanoprost, niacin, rosiglitazone, tamoxifen, canthaxanthine, sildenafil, interferon, chronic use of minocycline).

### Schedule of Activities

The Schedule of Activities is provided in Table 19

TABLE 19
Schedule of Activities

| | | | | Post-study |
|---|---|---|---|---|
| | Screening | | | (10 ± 2 days post |
| | (Days −28 | | | final dose)/Early |
| | to −2) | Day −1 | Days 1 to 22 | Termination |
| Adverse event questioning | | X | Ongoing | X |
| Vital signs (supine) ^{a} and 12-lead ECG | X | | Day 1: Predose, 1, 2, 4, and 6 hours postdose | X |
| | | | Day 3: Predose, 2, 4, 6, and 12 hours postdose | |
| | | | Day 7: Predose | |
| | | | Day 15: Predose, 1, 2, 4, and 6 hours postdose | |
| | | | Day 16: Predose | |
| | | | Day 18: 48 hours after last study drug administration | |
| | | | Day 22 | |
| Clinical laboratory evaluations (refer to | X | X | Day 3: Predose | X |
| Table 2) | | | Day 7: Predose | |
| | | | Day 15: Predose | |
| | | | Day 18: 48 hours after last study drug administration | |
| | | | Day 22 | |
| Blood sampling for midazolam PK | | | Day 1 and Day 15: Predose, 0.5, 1, 1.5, 2, 3, 4, 6, 8, 10, | |
| | | | 12, and 24 hours postdose | |
| Blood sampling for risdiplam PK | | | Day 3: Predose, 0.5, 1, 2, 3, 4, 5, 6, 8, 10, and 12 hours | |
| | | | postdose | |
| | | | Day 4 to Day 15: Predose | |
| | | | Day 16: Predose, 0.5, 1, 2, 3, 4, 5, 6, 8, 10, 12, 24, 36, 48, | |
| | | | 96, and 144 hours postdose | |

Abbreviations: ECG = electrocardiogram; PK = pharmacokinetic.
Note:
Nominal timepoints refer to the timepoint of risdiplam dose administration, with the exception of midazolam PK sampling and vital signs and ECG on Day 1, which refer to the timepoint of midazolam dose administration. The midazolam PK sample at 1 hour postdose corresponds to the same time of day as the risdiplam 2-hour postdose sample.
^{a} Systolic and diastolic blood pressure, pulse rate, and oral body temperature (oral body temperature at Screening and Day 1 predose only).

### Dose-Decision Criteria

The decision to proceed to Part 2 will be made following review of all safety and tolerability information collected up to 48 hours after last study drug administration (including AEs, ECGs, vital signs, and clinical laboratory test results), and of all PK data collected up to (and including) 24 hours after last study drug administration in Part 1 from a minimum of 4 participants. The dose of risdiplam to be administered in Part 2 will be selected to target a mean AUCtau at steady state of 2000 ng·h/mL (the therapeutic exposure observed in patients with SMA). The dose to be administered in Part 2 may only be greater than in Part 1 if the dose of 5 mg of risdiplam tested in Part 1 was safe and well tolerated and stopping rules were not met.

The decision to proceed to Part 2 will be made jointly by the Sponsor Clinical Pharmacologist and the Investigator and any other person(s) they consider necessary to assist with the decision.

The maximum possible dose for Part 2 is 18 mg of risdiplam, and this dose will not be exceeded under any circumstances.

### Stopping Rules Criteria

The dose of risdiplam in Part 2 will not be increased beyond 5 mg, if 1 of the following circumstances occurs in participants treated with 5 mg risdiplam in Part 1, unless it is obvious that the occurrence is not related to the administration of risdiplam.
Severe AEs of the same type in ≥50% of participants.
Clinically significant laboratory abnormalities of the same type in ≥50% of participants.
Clinically significant changes in ECGs of the same type in ≥50% participants.
Other findings, which at the joint discretion of the Sponsor Clinical Pharmacologist and the Investigator, indicate that the dose in Part 2 should not be increased.

### Individual Stopping Rules

Dosing will be stopped in a given individual participant if, compared to baseline (as applicable), 1 of the following circumstances occurs, unless it is obvious that the occurrence is not related to the administration of risdiplam:
An SAE.
Any elevation of alanine aminotransferase (ALT)>3×upper limit of normal (ULN),

with an associated increase in bilirubin >2×ULN, and with aspartate aminotransferase (AST)<2×ULN, in the absence of an alternative explanation.
Other findings that, at the joint discretion of the Sponsor Clinical Pharmacologist and the Investigator, indicate that dosing should be stopped.

### Lifestyle Considerations

### Meals and Dietary Restrictions

While confined at the Clinical Research Unit, participants will receive a standardized diet at scheduled times that do not conflict with other study-related activities. Participants will be fasted overnight (at least 8 hours) before collection of blood samples for clinical laboratory evaluations.

Participants will be fasted overnight (at least 8 hours) prior to dosing on Day 1 (Part 1) and on Days 1, 3, and 15 (Part 2) and will refrain from consuming water from 1 hour predose until 2 hours postdose, excluding the amount of water consumed at dosing. Food is allowed from 4 hours postdose. At all other times during the study, participants may consume water ad libitum. Foods and beverages containing poppy seeds will not be allowed from 7 days prior to Check-in (Day −1) and throughout the study (until after the Follow-up visit).

Foods and beverages containing grapefruit/grapefruit juice or Seville oranges will not be allowed from 14 days prior to study drug administration (Day 1) and throughout the study (until after the Follow-up visit).

Caffeine-containing foods and beverages will not be allowed from 48 hours before Check-in (Day −1) until discharge on Day 14.

Consumption of alcohol will not be permitted from 48 hours prior to Check-in (Day −1) until the Follow-up visit.

### Exercise

Participants are required to refrain from strenuous exercise from 7 days before Check-in (Day −1) until the Follow-up visit and will otherwise maintain their normal level of physical activity during this time (i.e., will not begin a new exercise program nor participate in any unusually strenuous physical exertion).

Participants may participate in light recreational activities during studies (e.g., watching television, reading).

### Safety Assessments

Planned timepoints for all safety assessments are provided in the SoA (Table 19).

Safety assessments will consist of monitoring and recording AEs, including SAEs and AEs of special interest (AESIs); measurement of protocol-specified safety laboratory assessments, vital signs, and ECGs; and other protocol-specified tests that are deemed critical to the safety evaluation of the study.

### Physical Examinations

A complete physical examination will include, at a minimum, assessments of the cardiovascular, respiratory, GI, dermatological, and musculoskeletal systems in addition to the head, eyes, ears, nose, throat, neck, and lymph nodes. Height, weight, and BMI will also be calculated and recorded at specified times. Further examination of other body systems may be performed in case of evocative symptoms at the Investigator's discretion.
A brief physical examination will include, at a minimum, assessments of the skin, lungs, cardiovascular system, and abdomen (liver and spleen).
Investigators should pay special attention to clinical signs related to previous serious illnesses.

The physical exam will NOT include pelvic, rectal, or breast exams.

Any abnormality identified at baseline should be recorded on the General Medical History and Baseline Conditions eCRF.

As clinically indicated, limited, symptom-directed physical examinations should be performed. Changes from baseline abnormalities should be recorded in the participant's notes. New or worsened clinically significant abnormalities should be recorded as AEs on the Adverse Event eCRF.

### Vital Signs

Temperature, PR, and systolic and diastolic blood pressure will be assessed as outlined in the SoA (Table 19).

Blood pressure and pulse measurements will be assessed in a supine position with a completely automated device. Manual techniques will be used only if an automated device is not available. When possible, the same arm and device should be used for all blood pressure measurements.

Blood pressure and pulse measurements should be preceded by at least 5 minutes of rest for the participant in a quiet setting without distractions (e.g., television, cell phones).

### Electrocardiograms

Single 12-lead ECGs will be obtained as outlined in the SoA (Table 19) using an ECG machine that automatically calculates the heart rate and measures PR, QRS, QT, and QTc intervals.

To minimize variability, it is important that participants be in a resting position for

≥10 minutes prior to each ECG evaluation. Supine body position should be consistently maintained for each ECG evaluation to prevent changes in heart rate. Environmental distractions (e.g., television, radio, conversation) should be avoided during the pre-ECG resting period and during ECG recording. Electrocardiograms should be performed prior to any scheduled vital signs measurements and blood draws.

### Clinical Safety Laboratory Assessments

A list of clinical laboratory tests to be performed is provided in Table 20 and these assessments must be conducted in accordance with the SoA (Table 19).

TABLE 20
Protocol-Required Safety Laboratory Assessments

Serum biochemistry:
Aspartate aminotransferase (AST)
Alanine aminotransferase (ALT)
Alkaline phosphatase
Gamma-glutamyl transferase (GGT)
Sodium
Potassium
Chloride
Calcium
Inorganic phosphate
Glucose
Urea
Bilirubin (Total and Direct)
Creatinine
Total protein
Albumin
Cholesterol
Triglycerides
Thyroid-stimulating hormone (TSH) ^{c}
Hematology:
White blood cell count (WBC)
Red blood cell count (RBC)
Hemoglobin
Hematocrit
Platelet count
Differential WBC (basophils, neutrophils,
eosinophils, monocytes, and lymphocytes)
Urinalysis:
Microscopic examination (sediment, RBCs,
WBCs, casts, crystals, epithelial cells,
bacteria), if blood or protein is abnormal.
pH
Protein
Glucose
Blood
Urinary drug screen:
Drugs of abuse ^{a}
Hormone panel: ^{b,c}
Follicle-stimulating hormone (FSH) ^{d}
Estradiol ^{d}
Human chorionic gonadotropin (hCG) (serum
pregnancy test)
Serology: ^{c}
Hepatitis B surface antigen (HBsAg)
Hepatitis C antibody
Human immunodeficiency virus (HIV)
antibodies
Coagulation: ^{c}
International normalized ratio (INR)
Activated partial thromboplastin time (aPTT)
Prothrombin time (PT)

^{a} Opiates, amphetamines, cannabinoids, benzodiazepines, cocaine, barbiturates, methadone, cotinine, and alcohol.
^{b} Females only.
^{c} Analyzed at Screening only.
^{d} Post-menopausal females only.

### Pharmacokinetics

Mandatory blood samples to evaluate concentrations of study treatment (and its metabolite[s], if appropriate) will be collected. The date and time of each sample collection will be recorded in the eCRF. Risdiplam and midazolam (Part 2 only) levels will be analyzed by using validated assays. The PK samples will be taken as outlined in the Schedules of Activities tables (see Table 20). During the course of the study, PK sampling timepoints may be modified on the basis of emerging data to ensure the PK of study treatment can be adequately characterized. Metabolites may be measured by a specific validated liquid chromatography with tandem mass spectrometry assay, or other fit for purpose methods as appropriate.

The PK blood samples will be destroyed after the date of final Clinical Study Report or after approval of sample destruction by the study management team. Details on sampling procedures, sample storage, and shipment are given in the sample documentation.

Any changes in the timing or addition of PK timepoints must be documented and approved by the relevant study team member and then archived in the Sponsor and site study files, but this will not constitute a protocol amendment.

### Statistical Analyses STATISTICAL ANALYSES

### Safety Analyses

All safety analyses will be based on the safety analysis population. Safety analyses are detailed in Table 21.

TABLE 21
Safety Statistical Analysis Methods

| Endpoint | Statistical Analysis Methods |
|---|---|
| Adverse | The original terms recorded on the eCRF by the |
| events | Investigator for adverse events will be coded by the |
| | Sponsor. |
| | Adverse events will be summarized by mapped term |
| | and appropriate thesaurus level. |
| Clinical | All clinical laboratory data will be stored on the |
| laboratory | database in the units in which they were reported. |
| tests | Laboratory test values will be presented in |
| | International System of Units (SI units; Système |
| | International d'Unités) by individual listings with |
| | flagging of abnormal results. Summaries of |
| | clinical laboratory tests will also be used, as |
| | appropriate. |
| Vital signs | Vital signs data will be presented by individual listings |
| | with flagging of values outside the normal ranges and |
| | flagging of abnormalities. In addition, tabular |
| | summaries will be used, as appropriate. |
| ECG data | ECG data will be presented by individual listings. In |
| analysis | addition, tabular summaries will be used, as |
| | appropriate. |
| Concomitant | The original terms recorded on the participants' eCRF |
| medications | by the Investigator for concomitant medications will be |
| | standardized by the Sponsor by utilizing a mapped term |
| | and appropriate drug dictionary level. |

### Pharmacokinetic Analyses

Analyses will be carried out on the PK analysis population. All PK parameters will be presented by listings and descriptive summary statistics (arithmetic mean, standard deviation, geometric mean, geometric coefficient of variation, median, minimum, and maximum). For Tmax, only the median, minimum, and maximum values will be presented.

Pharmacokinetic parameters will be read directly from the plasma concentration-time profiles, or calculated using standard non-compartmental methods.

The following PK parameters will be computed for risdiplam and its metabolite(s) as appropriate and midazolam and its metabolite(s) as appropriate. Other PK parameters might be computed in addition as appropriate.
Tₘₐₓ Time of maximum observed plasma concentration
Cₘₐₓ Maximum observed plasma concentration
C_{trough} Trough observed plasma concentration
AUCₜₐᵤ Area under the plasma concentration-time curve over a dosing interval
AUCₗₐₛₜ Area under the plasma concentration-time curve from time 0 to the time of last quantifiable concentration (tₗₐₛₜ)
AUC_{inf} Area under the plasma concentration-time curve extrapolated to infinity
λ_{z} Apparent terminal elimination rate constant
t_{1/2} Apparent plasma terminal elimination half-life
CLₛₛ/F Apparent total plasma clearance at steady state
AR_{AUC} Accumulation ratio for AUC
AR_{Cmax} Accumulation ratio for Cmax

In Part 2, the effect of multiple oral doses of risdiplam on the PK of a single oral dose of midazolam (and its metabolite[s] as appropriate) will be explored using an analysis of variance applied to the log-transformed PK parameters Cₘₐₓ and AUC_{inf} (or, if AUC_{inf} cannot be properly estimated, AUCₗₐₛₜ or an alternate partial AUC from time zero to a common postdose time, AUC₀₋ₜ). The model will include treatment as a fixed effect and subject as a random effect. From the model estimates, the geometric mean ratios (midazolam alone versus midazolam in combination with risdiplam) will be derived together with corresponding two-sided 90% confidence intervals.

### REFERENCES

1) Obach R S and Walsky R L (2004) Validated assays for human cytochrome P450 activities. Drug Metab Dispos: 32:647
2) Obach R S, Walsky R L and Venkatakrishnan K (2007) Mechanism-based inactivation of human cytochrome P450 enzymes and the prediction of drug-drug interactions. Drug Metab Dispos 35:246
3) Perloff E S, Mason A K, Dehal S S, Blanchard A P, Morgan L, Ho T, Dandeneau A, Crocker R M, Chandler C M, Boily N, Crespi C L, and Stresser D M (2009) Validation of Cytochrome P450 Time Dependent Inhibition Assays: A Two Time Point IC50 Shift Approach Facilitates kinact Assay Design. Xenobiotica 39:99

1-56. (canceled)

57. A method for the treatment of SMA in a subject being treated with concomitant administration of a CYP3A substrate, said method comprising administering to said subject a therapeutically effective amount of risdiplam while reducing the concomitant administration of a CYP3A substrate.

58. The method of claim 57, wherein the subject is a human.

59. The method of claim 57, wherein the therapeutically effective amount of risdiplam is a total daily dose of
0.2 mg/kg for subjects between 2 months and 2 years of age,
0.25 mg/kg for subjects older than 2 years of age and with a body weight of less than kg, or
5 mg for subjects older than 2 years of age and with a body weight of more than or equal to 20 kg.

60. The method of claim 59, wherein the risdiplam is administered orally at a total daily dose of 0.2 mg/kg for subjects between 2 months and 2 years of age.

61. The method of claim 59, wherein the risdiplam is administered orally at a total daily dose of 0.25 mg per kilogram of body weight for a subject older than 2 years of age and with a body weight of less than 20 kg.

62. The method of claim 59, wherein the risdiplam is administered orally at a total daily dose of 5 mg for a subject older than 2 years of age and with a body weight of more than or equal to 20 kg.

63. The method of claim 57, wherein the subject has type I SMA, type II SMA or type III SMA.

64. The method of claim 57, wherein reducing concomitant administration of a CYP3A substrate is to avoid the potential for a reduced clearance of the CYP3A substrate or the potential for an increased exposure to the CYP3A substrate.

65. The method of claim 57, wherein reducing concomitant administration of a CYP3A substrate comprises reducing the dosage of CYP3A substrate.

66. The method of claim 65, wherein the dosage of CYP3A substrate is reduced by 10%, 15%, 20%, 25%, 30%, 35%, 40%, 45%, 50%, 55% 60%, 65%, 70%, 75%, 80%, 85%, 90%, 95% or 98% of the standard dosage of CYP3A substrate during concomitant administration of risdiplam.

67. The method of claim 66, wherein the dosage of CYP3A substrate is reduced by 10% or 15% of the standard dosage of CYP3A substrate during concomitant administration of risdiplam.

68. The method of claim 65, wherein the reduced dosage of CYP3A substrate is the normal prescribed dosage of CYP3A substrate.

69. The method of claim 57, wherein the CYP3A substrate is midazolam.

70. The method of claim 69, wherein midazolam is being administered as an oral, intravenous, intramuscular or rectal route of administration.

71. The method of claim 69, wherein the midazolam is administered orally in unit dosage forms that are capsules or tablets.

72. The method of claim 71, wherein the amount of midazolam in the unit dosage form is 7.5 mg or 15 mg.

73. The method of claim 69, wherein the midazolam is being administered to the subject for sedative therapy or for treatment of sleep disturbance or for seizures.

74. A pharmaceutical composition comprising risdiplam, a CYP3A substrate in an amount reduced dose by 10%, 15%, 20%, 25%, 30%, 35%, 40%, 45%, 50%, 55%, 60%, 65%, 70%, 75%, 80%, 85%, 90%, 95% or 98% of the standard dosage of the CYP3A substrate, and a pharmaceutically acceptable excipient.

75. The pharmaceutical composition of claim 74, wherein the CYP3A substrate is midazolam.

76. The pharmaceutical composition of claim 74, wherein the pharmaceutical composition comprises risdiplam as a dry powder or granulation for constitution of an oral solution.
